SEQUENCE LISTING (21 sequences)

SEQ ID NO: 1 - DNA, 990 bp, synthetic construct
  source 1..990: mol_type = other DNA
atgagcttctcacatgacacccctgaaatcgtttacacccacgacaccggtctggactatatcacctactctgactacgaactggacccggctaacccgctggctggtggtgctgcttggatcgaaggtgctttcgttccgccgtctgaagctcgtatctctatcttcgaccagggtttttatacttctgacgctacctacaccgtcttccacgtttggaacggtaacgctttccgtctgggggaccacatcgaacgtctgttctctaatgcggaatctattcgtttgatcccgccgctgacccaggacgaagttaaagagatcgctctggaactggttgctaaaaccgaactgcgtgaagcgatcgttatcgtttctatcacccgtggttactcttctaccccatgggagcgtgacatcaccaaacatcgtccgcaggtttacatgtatgctgttccgtaccagtggatcgtaccgtttgaccgcatccgtgacggtgttcacctgatggttgctcagtcagttcgtcgtacaccgcgtagctctatcgacccgcaggttaaaaacttccagtggggtgacctgatccgtgcaattcaggaaacccacgaccgtggtttcgagttaccgctgctgctggacttcgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggctgacatcaccccggctgaactgtacgacgctgacgaagttctgggttgctcaaccggtggtggtgtttggccgttcgtttctgttgacggtaactctatctctgacggtgttccgggtccggttacccagtctatcatccgtcgttactgggaactgaacgttgaaccttcttctctgctgaccccggtacagtac

SEQ ID NO: 2 - protein, 330 aa, synthetic construct
MSFSHDTPEIVYTHDTGLDYITYSDYELDPANPLAGGAAWIEGAFVPPSEARISIFDQGFYTSDATYTVFHVWNGNAFRLGDHIERLFSNAESIRLIPPLTQDEVKEIALELVAKTELREAIVIVSITRGYSSTPWERDITKHRPQVYMYAVPYQWIVPFDRIRDGVHLMVAQSVRRTPRSSIDPQVKNFQWGDLIRAIQETHDRGFELPLLLDFDNLLAEGPGFNVVVIKDGVVRSPGRAALPGITRKTVLEIAESLGHEAILADITPAELYDADEVLGCSTGGGVWPFVSVDGNSISDGVPGPVTQSIIRRYWELNVEPSSLLTPVQY

SEQ ID NO: 3 - DNA, 990 bp, synthetic construct
  source 1..990: mol_type = other DNA
atgagcttctcacatgacacccctgaaatcgtttacacccacgacaccggtctggactatatcacctactctgactacgaactggacccggctaacccgctggctggtggtgctgcttggatcgaaggtgctttcgttccgccgtctgaagctcgtatctctatcttcgaccagggtttttgtacttctgacgctacctacaccgtcttccacgtttggaacggtaacgctttccgtctgggggaccacatcgaacgtctgttctctaatgcggaatctattcgtttgatcccgccgctgacccaggacgaagttaaagagatcgctctggaactggttgctaaaaccgaactgcgtgaagcgatcgttatcgtttctatcacccgtggttactcttctaccccatgggagcgtgacatcaccaaacatcgtccgcaggtttacatgtatgctgttccgtaccagtggatcgtaccgtttgaccgcatccgtgacggtgttcacctgatggttgctcagtcagttcgtcgtacaccgcgtagctctatcgacccgcaggttaaaaacttccagtggggtgacctgatccgtgcaattcaggaaacccacgaccgtggtttcgagttaccgctgctgctggacttcgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggctgacatcaccccggctgaactgtacgacgctgacgaagttctgggttgctcaaccggtggtggtgtttggccgttcgtttctgttgacggtaactctatctctgacggtgttccgggtccggttacccagtctatcatccgtcgttactgggaactgaacgttgaaccttcttctctgctgaccccggtacagtac

SEQ ID NO: 4 - protein, 330 aa, synthetic construct
MSFSHDTPEIVYTHDTGLDYITYSDYELDPANPLAGGAAWIEGAFVPPSEARISIFDQGFCTSDATYTVFHVWNGNAFRLGDHIERLFSNAESIRLIPPLTQDEVKEIALELVAKTELREAIVIVSITRGYSSTPWERDITKHRPQVYMYAVPYQWIVPFDRIRDGVHLMVAQSVRRTPRSSIDPQVKNFQWGDLIRAIQETHDRGFELPLLLDFDNLLAEGPGFNVVVIKDGVVRSPGRAALPGITRKTVLEIAESLGHEAILADITPAELYDADEVLGCSTGGGVWPFVSVDGNSISDGVPGPVTQSIIRRYWELNVEPSSLLTPVQY

SEQ ID NO: 5 - DNA, 990 bp, synthetic construct
  source 1..990: mol_type = other DNA
atgagcttctcacatgacacccctgaaatcgtttacacccacgacaccggtctggactatatcacctactctgactacgaactggacccggctaacccgctggctggtggtgctgcttggatcgaaggtgctttcgttccgagatctgaagctcgtatctctatcttcgaccagggtttttgtacttctgacgctacctacacctgcttccacgtttggaacggtaacgctttccgtctgggggaccacatcgaacgtctgttctctaatgcggaatcttgtcgtttgatcccgccgctgacccaggacgaagttaaagagatcgctctggaactggttgctaaaaccgaactgcgtgaagcgatcgttatcgtttctatcacccgtggttactcttctaccccatgggagcgtgacatcaccaaacatcgtccgcaggtttacatgtatgctgttccgtaccagtggatcgtaccgtttgaccgcatccgtgacggtgttcacctgatggttgctcagtcagttcgacgtacaccgcgtagctctatcgacccgcaggttaaaaacttccagtggggtgacctgagacgtgcaattcaggaaacccacgaccgtggtttcgagttaccgctgctgctggacttcgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggccgacatcaccccggctgaactgtacgacgctgacgaagttctgggttgctcaaccggtggtggtgtttggccgttcgtttctgttgacggtaaccgcatctctgacggtgttccgggtccggttacccagtctatcatccgtcgttactgggaactgaacgttgaaccttcttctctgctgaccccggtacagtac

SEQ ID NO: 6 - protein, 330 aa, synthetic construct
MSFSHDTPEIVYTHDTGLDYITYSDYELDPANPLAGGAAWIEGAFVPRSEARISIFDQGFCTSDATYTCFHVWNGNAFRLGDHIERLFSNAESCRLIPPLTQDEVKEIALELVAKTELREAIVIVSITRGYSSTPWERDITKHRPQVYMYAVPYQWIVPFDRIRDGVHLMVAQSVRRTPRSSIDPQVKNFQWGDLRRAIQETHDRGFELPLLLDFDNLLAEGPGFNVVVIKDGVVRSPGRAALPGITRKTVLEIAESLGHEAILADITPAELYDADEVLGCSTGGGVWPFVSVDGNRISDGVPGPVTQSIIRRYWELNVEPSSLLTPVQY

SEQ ID NO: 7 - DNA, 990 bp, synthetic construct
  source 1..990: mol_type = other DNA
atgagcttctcacatgacacccctgaaatcgtttacacccacgacaccggtctggactatatcacctactctgactacgaactggacccggctaacccgctggctggtggtgctgcttggatcgaaggtgctttcgttccgagatctgaagctcgtatctctatcttcgaccagggttttaatacttctgacgctacctacacctgcttccacgtttggaacggtaacgctttccgtctgggggaccacatcgaacgtctgttctctaatgcggaatcttgtcgtttgatcccgccgctgacccaggacgaagttaaagagatcgctctggaactggttgctaaaaccgaactgcgtgaagcgatcgttatcgtttctatcacccgtggttactcttctaccccatgggagcgtgacatcaccaaacatcgtccgcaggtttacatgtatgctgttccgtaccagtggatcgtaccgtttgaccgcatccgtgacggtgttcacctgatggttgctcagtcagttcgacgtacaccgcgtagctctatcgacccgcaggttaaaaacttccagtggggtgacctgagacgtgcaattcaggaaacccacgaccgtggtttcgagttaccgctgctgctggacttcgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggccgacatcaccccggctgaactgtacgacgctgacgaagttctgggttgctcaaccggtggtggtgtttggccgttcgtttctgttgacggtaaccgcatctctgacggtgttccgggtccggttacccagtctatcatccgtcgttactgggaactgaacgttgaaccttcttctctgctgaccccggtacagtac

SEQ ID NO: 8 - protein, 330 aa, synthetic construct
MSFSHDTPEIVYTHDTGLDYITYSDYELDPANPLAGGAAWIEGAFVPRSEARISIFDQGFNTSDATYTCFHVWNGNAFRLGDHIERLFSNAESCRLIPPLTQDEVKEIALELVAKTELREAIVIVSITRGYSSTPWERDITKHRPQVYMYAVPYQWIVPFDRIRDGVHLMVAQSVRRTPRSSIDPQVKNFQWGDLRRAIQETHDRGFELPLLLDFDNLLAEGPGFNVVVIKDGVVRSPGRAALPGITRKTVLEIAESLGHEAILADITPAELYDADEVLGCSTGGGVWPFVSVDGNRISDGVPGPVTQSIIRRYWELNVEPSSLLTPVQY

SEQ ID NO: 9 - DNA, 990 bp, synthetic construct
  source 1..990: mol_type = other DNA
atgagcttctcacatgacacccctgaaatcgtttacacccacgacaccggtctggactatatcacctactctgactacgaactggacccggctaacccgctggctggtggtgctgcttggatcgaaggtgctttcgttccgagatctgaagctcgtatctctatcttcgaccagggttttaatacttctgacgctacctacaccacattccacgtttggaacggtaacgctttccgtctgggggaccacatcgaacgtctgttctctaatgcggaatcttgtcgtttgatcccgccgctgacccaggacgaagttaaagagatcgctctggaactggttgctaaaaccgaactgcgtgaagcgatcgttatcgtttctatcacccgtggttactcttctaccccatgggagcgtgacatcaccaaacatcgtccgcaggtttacatgtatgctgttccgtaccagtggatcgtaccgtttgaccgcatccgtgacggtgttcacctgatggttgctcagtcagttcgacgtacaccgcgtagctctatcgacccgcaggttaaaaacttccagtggggtgacctgagacgtgcaattcaggaaacccacgaccgtggtttcgagttaccgctgctgctggacttcgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggccgacatcaccccggccgaactgtacgacgctgacgaagttctgggttgctcaaccggtggtggtgtttggccgttcgtttctgttgacggtaaccgcatctctgacggtgttccgggtccggttacccagtctatcatccgtcgttactgggaactgaacgttgaaccttcttctctgctgaccccggtacagtac

SEQ ID NO: 10 - protein, 330 aa, synthetic construct
MSFSHDTPEIVYTHDTGLDYITYSDYELDPANPLAGGAAWIEGAFVPRSEARISIFDQGFNTSDATYTTFHVWNGNAFRLGDHIERLFSNAESCRLIPPLTQDEVKEIALELVAKTELREAIVIVSITRGYSSTPWERDITKHRPQVYMYAVPYQWIVPFDRIRDGVHLMVAQSVRRTPRSSIDPQVKNFQWGDLRRAIQETHDRGFELPLLLDFDNLLAEGPGFNVVVIKDGVVRSPGRAALPGITRKTVLEIAESLGHEAILADITPAELYDADEVLGCSTGGGVWPFVSVDGNRISDGVPGPVTQSIIRRYWELNVEPSSLLTPVQY

SEQ ID NO: 11 - DNA, 990 bp, synthetic construct
  source 1..990: mol_type = other DNA
atgagcttctcacatgacacccctgaaatcgtttacacccacgacaccggtctggactatatcacctactctgactacgaactggacccggctaacccgctggctggtggtgctgcttggatcgaaggtgctttcgttccgagatctgaagctcgtatctctatcttcgaccagggttttaatacttctgacgctacctacaccacattccacgtttggaacggtaacgctttccgtctgggggaccacatcgaacgtctgttctctaatgcggaatcttgtcgtttgatcccgccgctgacccaggacgaagttaaagagatcgctctggaactggttgctaaaaccgaactgcgtgaagcgatcgttcatgtttctatcacccgtggttactcttctaccccatgggagcgtgacatcaccaaacatcgtccgcaggtttacatgtatgctgttccgtaccagtggatcgtaccgtttgaccgcatccgtgacggtgttcacctgatggttgctcagtcagttcgacgtacaccgcgtagctctatcgacccgcaggttaaaaacttccagtggggtgacctgagacgtgcaattcaggaaacccacgaccgtggtttcgagttaccgctgctgctggacttcgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggccgacatcaccccggccgaactgtacgacgctgacgaagttctgggttgctcaaccggtggtggtgtttggccgttcgtttctgttgacggtaaccgcatctctgacggtgttccgggtccggttacccagtctatcatccgtcgttactgggaactgaacgttgaaccttcttctctgctgaccccggtacagtac

SEQ ID NO: 12 - protein, 330 aa, synthetic construct
MSFSHDTPEIVYTHDTGLDYITYSDYELDPANPLAGGAAWIEGAFVPRSEARISIFDQGFNTSDATYTTFHVWNGNAFRLGDHIERLFSNAESCRLIPPLTQDEVKEIALELVAKTELREAIVHVSITRGYSSTPWERDITKHRPQVYMYAVPYQWIVPFDRIRDGVHLMVAQSVRRTPRSSIDPQVKNFQWGDLRRAIQETHDRGFELPLLLDFDNLLAEGPGFNVVVIKDGVVRSPGRAALPGITRKTVLEIAESLGHEAILADITPAELYDADEVLGCSTGGGVWPFVSVDGNRISDGVPGPVTQSIIRRYWELNVEPSSLLTPVQY

SEQ ID NO: 13 - DNA, 990 bp, synthetic construct
  source 1..990: mol_type = other DNA
atgagcttctcacatgacacccctgaaatcgtttacacccacgacaccggtctggactatatcacctactctgactacgaactggacccggctaacccgctggctggtggtgctgcttggatcgaaggtgctttcgttccgagatctgaagctcgtatctctatcttcgaccagggttttatgacttctgacgctacctacaccacattccacgtttggaacggtaacgctttccgtctgggggaccacatcgaacgtctgttctctaatgcggaatcttgtcgtttgatcccgccgctgacccaggacgaagttaaagagatcgctctggaactggttgctaaaaccgaactgcgtgaagcgatcgttatcgtttctatcacccgtggttactcttctaccccatgggagcgtgacatcaccaaacatcgtccgcaggtttacatgtatgctgttccgtaccagtggatcgtaccgtttgaccgcatccgtgacggtgttcacctgatggttgctcagtcagttcgacgtacaccgcgtagctctatcgacccgcaggttaaaaacttccagtggggtgacctgagacgtgcaattcaggaaacccacgaccgtggtttcgagttaccgctgctgctggacttcgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggccgacatcaccccggccgaactgtacgacgctgacgaagttctgggttgctcaaccggtggtggtgtttggccgttcgtttctgttgacggtaaccgcatctctgacggtgttccgggtccggttacccagtctatcatccgtcgttactgggaactgaacgttgaaccttcttctctgctgaccccggtacagtac

SEQ ID NO: 14 - protein, 330 aa, synthetic construct
MSFSHDTPEIVYTHDTGLDYITYSDYELDPANPLAGGAAWIEGAFVPRSEARISIFDQGFMTSDATYTTFHVWNGNAFRLGDHIERLFSNAESCRLIPPLTQDEVKEIALELVAKTELREAIVIVSITRGYSSTPWERDITKHRPQVYMYAVPYQWIVPFDRIRDGVHLMVAQSVRRTPRSSIDPQVKNFQWGDLRRAIQETHDRGFELPLLLDFDNLLAEGPGFNVVVIKDGVVRSPGRAALPGITRKTVLEIAESLGHEAILADITPAELYDADEVLGCSTGGGVWPFVSVDGNRISDGVPGPVTQSIIRRYWELNVEPSSLLTPVQY

SEQ ID NO: 15 - DNA, 990 bp, synthetic construct
  source 1..990: mol_type = other DNA
atgagcttctcacatgacacccctgaaatcgtttacacccacgacaccggtctggactatatcacctactctgactacgaactggacccggctaacccgctggctggtggtgctgcttggatcgaaggtgctttcgttccgagatctgaagctcgtatctctatcttcgaccagggttttatgacttctgacgctacctacaccacattccacgtttggaacggtaacgctttccgtctgggggaccacatcgaacgtctgttctctaatgcggaatcttgtcgtttgatcccgccgctgacccaggacgaagttaaagagatcgctctggaactggttgctaaaaccgaactgcgtgaagcgatcgttatcgtttctatcacccgtggttactcttctaccccatggacacgtgacatcaccaaacatcgtccgcaggtttacatgtatgctgttccgtaccagtggatcgtaccgtttgaccgcatccgtgacggtgttcacctgatggttgctcagtcagttcgacgtacaccgcgtagctctatcgacccgcaggttaaaaacttccagtggggtgacctgagacgtgcaattcaggaaacccacgaccgtggtttcgagttaccgctgctgctggacttcgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggccgacatcaccccggccgaactgtacgacgctgacgaagttctgggttgctcaaccggtggtggtgtttggccgttcgtttctgttgacggtaaccgcatctctgacggtgttccgggtccggttacccagtctatcatccgtcgttactgggaactgaacgttgaaccttcttctctgctgaccccggtacagtac

SEQ ID NO: 16 - protein, 330 aa, synthetic construct
MSFSHDTPEIVYTHDTGLDYITYSDYELDPANPLAGGAAWIEGAFVPRSEARISIFDQGFMTSDATYTTFHVWNGNAFRLGDHIERLFSNAESCRLIPPLTQDEVKEIALELVAKTELREAIVIVSITRGYSSTPWTRDITKHRPQVYMYAVPYQWIVPFDRIRDGVHLMVAQSVRRTPRSSIDPQVKNFQWGDLRRAIQETHDRGFELPLLLDFDNLLAEGPGFNVVVIKDGVVRSPGRAALPGITRKTVLEIAESLGHEAILADITPAELYDADEVLGCSTGGGVWPFVSVDGNRISDGVPGPVTQSIIRRYWELNVEPSSLLTPVQY

SEQ ID NO: 17 - DNA, 990 bp, synthetic construct
  source 1..990: mol_type = other DNA
atgagcttctcacatgacacccctgaaatcgtttacacccacgacaccggtctggactatatcacctactctgactacgaactggacccggctaacccgctggctggtggtgctgcttggatcgaaggtgctttcgttccgagatctgaagctcgtatctctatcttcgaccagggttttatgacttctgacgctacctacaccacattccacgtttggaacggtaacgctttccgtctgggggaccacatcgaacgtctgttctctaatgcggaatcttgtcgtttgatcccgccgctgacccaggacgaagttaaagagatcgctctggaactggttgctaaaaccgaactgcgtgaagcgatcgttattgtttctatcacccgtggttactcttctaccccatggacacgcgaccaaaccaaacatcgtccgcaggtttacatgtatgctgttccgtaccagtggattgtaccgtttgaccgcatccgtgacggtgttcatctgatggttgctcagtcagttcgacgtacaccgcgtagctctatcgacccgcaggttaaaaacttccagtggggtgacctgagacgtgcaacccaggaatgtcacgaccgtggtttcgagttaccgctgctgctggacttcgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggccgacatcaccccggccgaactgtacgacgctgacgaagttctgggttgctcaaccggtggtggtgtttggccgttcgtttctgttgacggtaaccgcatctctgacggtgttccgggtccggttacccagtctatcatccgtcgttactgggaactgaacgttgaaccttcttctctgctgaccccggtacagtac

SEQ ID NO: 18 - protein, 330 aa, synthetic construct
MSFSHDTPEIVYTHDTGLDYITYSDYELDPANPLAGGAAWIEGAFVPRSEARISIFDQGFMTSDATYTTFHVWNGNAFRLGDHIERLFSNAESCRLIPPLTQDEVKEIALELVAKTELREAIVIVSITRGYSSTPWTRDQTKHRPQVYMYAVPYQWIVPFDRIRDGVHLMVAQSVRRTPRSSIDPQVKNFQWGDLRRATQECHDRGFELPLLLDFDNLLAEGPGFNVVVIKDGVVRSPGRAALPGITRKTVLEIAESLGHEAILADITPAELYDADEVLGCSTGGGVWPFVSVDGNRISDGVPGPVTQSIIRRYWELNVEPSSLLTPVQY

SEQ ID NO: 19 - DNA, 990 bp, synthetic construct
  source 1..990: mol_type = other DNA
atgagccactcaattgacacacctgaaatcgtttacacccacgacaccggtctggactatatcacctactctgactacgaactggacccggctaacccgctggctggtggtgctgcttggatcgaaggtgctttcgttccgagatctgaagctcgtatctctatcttcgaccagggttttatgacttctgacgctacctacaccacattccacgtttggaacggtaacgctttccgtctgggtgaccacatcgaacgtctgttctctaatgcggaatcttgtcgtttggagccgccgctgacccaggacgaagttaaagagatcgctctggaactggttgctaaaaccgaactgcgtgaagcgatcgttattgtttctatcacccgtggttactcttctaccccatggacacgcgaccaaaccaaacatcgtccgcaggtttacatgtatgctgttccgtaccagtggattgtaccgtttgaccgcatccgtgacggtgttcatctgatggttgctcagtcagttcgacgtacaccgcgtagctctatcgacccgcaggttaaaaacttccagtggggtgacctgagacgtgcaacccaggaatgtcacgaccgtggtttcgagttaccgctgctgctggacttcgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggccgacatcaccgtggccgaactgtacgacgctgacgaagttctgggttgctcaaccggtggtggtgtttggccgttcgtttctgttgacggtaaccgcatctctgacggtgttccgggtccggttacccagtctatcatccgtcgttactgggaactgaacgttgaaccttcttctctgctgaccccggtacagtac

SEQ ID NO: 20 - protein, 330 aa, synthetic construct
MSHSIDTPEIVYTHDTGLDYITYSDYELDPANPLAGGAAWIEGAFVPRSEARISIFDQGFMTSDATYTTFHVWNGNAFRLGDHIERLFSNAESCRLEPPLTQDEVKEIALELVAKTELREAIVIVSITRGYSSTPWTRDQTKHRPQVYMYAVPYQWIVPFDRIRDGVHLMVAQSVRRTPRSSIDPQVKNFQWGDLRRATQECHDRGFELPLLLDFDNLLAEGPGFNVVVIKDGVVRSPGRAALPGITRKTVLEIAESLGHEAILADITVAELYDADEVLGCSTGGGVWPFVSVDGNRISDGVPGPVTQSIIRRYWELNVEPSSLLTPVQY

SEQ ID NO: 21 - protein, 21 aa, synthetic construct
  VARIANT 2..21: note = This region may encompass 0-20 residues
MHHHHHHHHHHHHHHHHHHHH

(19) United States
(12) Patent Application Publication
(10) Pub. No.: US 2026/0337585 A1
(43) Pub. Date: Oct. 29, 2026

(54) BIOCATALYSTS AND METHODS THEREOF

(71) Applicant: Tesaro,, Inc., Wilmington, DE (US)

(72) Inventors: Katherine Joyce HONICKER, Collegeville, PA (US); Justin M. KAPLAN, Collegeville, PA (US); Jonathan LATHAM, Collegeville, PA (US); Joel MELBY, Collegeville, PA (US); Christophet W. MORGAN, Collegeville, PA (US); Qiaogong SU, Collegeville, PA (US)

(21) Appl. No.: 19/105,842

(22) PCT Filed: Aug. 24, 2023
(86) PCT No.: PCT/US2023/072775

§ 371 Date: Feb. 24, 2025

Related U.S. Application Data
(60) Provisional application No. 63/401,250, filed on Aug. 26, 2022.

(51) Int. Cl. C12N 9/10; C12P 17/10

(52) U.S. Cl. CPC C12N 9/1096; C12P 17/10

(57) ABSTRACT

The present disclosure relates to transaminase biocatalysts and methods of using the biocatalysts. The present disclosure also includes methods for preparing amine intermediate compounds useful for manufacturing niraparib. The present disclosure also includes polynucleotides encoding the transaminase biocatalysts and host cells comprising said polynucleotides.

### FIELD OF THE INVENTION

The present disclosure relates to transaminase biocatalysts and methods of using the biocatalysts. The present disclosure also includes methods for preparing amine intermediate compounds useful for manufacturing niraparib. The present disclosure also includes polynucleotides encoding the transaminase biocatalysts and host cells comprising said polynucleotides.

### BACKGROUND OF THE INVENTION

Poly (ADP-ribose) polymerases (PARP) are a family of enzymes involved in DNA damage repair and PARP inhibition leads to accumulation of single-strand breaks, which then leads to an accumulation of double strand breaks. Cells with an increasing number of DSBs are more dependent on other DNA repair pathways, mainly the homologous recombination (HR) repair pathway. Cancer cells with HR deficiencies are therefore even more susceptible to PARP impairment of the BER pathway. There are currently four approved PARP inhibitors (e.g., niraparib, olaparib, rucaparib and talazoparib), with many more currently in clinical trials.

Niraparib is currently approved as a PARP inhibitor for the maintenance treatment of adult patients with advanced or recurrent epithelial ovarian, fallopian tube, or primary peritoneal cancer who are in a complete or partial response to first-line platinum-based chemotherapy; and for the treatment of adult patients with advanced ovarian, fallopian tube, or primary peritoneal cancer who have been treated with three or more prior chemotherapy regimens and whose cancer is associated with homologous recombination deficiency (HRD) positive status defined by either a deleterious or suspected deleterious BRCA mutation, or genomic instability and who have progressed more than six months after response to the last platinum-based chemotherapy.

Transaminases, also known as aminotransferases, catalyze the transfer of an amino group, a pair of electrons, and a proton from a primary amine of an amino donor substrate to the carbonyl group (i.e., a keto group) of an amino acceptor molecule (Shin et al., 2001, Biosci. Biotechnol, Biochem. 65: 1782-1788).

The stereoselectivity of transaminases in the conversion of a ketone to the corresponding amine make these enzymes useful in the asymmetric synthesis of optically pure amines from the corresponding keto compounds (Hohne et al., Chem. Cat. Chem. 1(1):42-51). Transaminases can also be applied to the chiral resolution of racemic amines by exploiting the ability of the transaminases to carry out the reverse reaction in a stereospecific manner, i.e., preferential conversion of one enantiomer to the corresponding ketone, thereby resulting in a mixture enriched in the other enantiomer (Koselewski et al., 2009, Org. Lett. 1 1(21):4810-2). The wild-type transaminase from Arthrobacter sp. KNK168 is an R-selective enzyme that produces R-amines from some substrates. Some studies have shown that engineered transaminase polypeptides derived from the naturally occurring transaminase of Arthrobacter sp. KNK168 have increased stability to temperature and/or organic solvent, and which have been adapted to have enzymatic activity towards structurally different amino acceptor molecules (Savile et al., 2010, Science 329(5989): 305-9).

Various manufacturing processes for preparing niraparib have been developed over the years. However, there is a need for improved transaminase biocatalysts that can be used to prepare amine intermediate compounds useful for manufacturing niraparib and new processes employing those biocatalysts that are simple, cost effective, non-hazardous, and commercially viable.

### SEQUENCE LISTING

The instant application contains a Sequence Listing, which has been submitted electronically in computer readable form in an XML format and is hereby incorporated by reference in its entirety. Said XML file, created on Jun. 27, 2023, is named “TES00050WO01.xml” and is 32,471 bytes in size.

### SUMMARY OF THE INVENTION

The present invention provides engineered transaminase polypeptides having transaminase activity, polynucleotides encoding the polypeptides, methods of making the polypeptides, and methods of using the polypeptides for preparing amine intermediate compounds useful for manufacturing niraparib. In one aspect of the present invention, engineered transaminase polypeptides comprising at least one, at least two, at least three, at least four, at least five, at least ten, or at least twenty amino acid substitution and/or modification relative to a transaminase variant as set forth in SEQ ID NO: 2 are provided.

The present invention provides an engineered transaminase polypeptide comprising an amino acid sequence having at least 80%, 85%, 90%, or 95% sequence identity to the amino acid sequence set forth in SEQ ID NO: 2 or a functional fragment thereof, wherein the amino acid sequence comprises the feature that X124 is isoleucine (I).

In a further aspect of the present invention, polynucleotides encoding the engineered transaminase polypeptides of the present invention are provided. Also provided are compositions, vectors, and host cells comprising said polynucleotides.

In a further aspect, the present invention provides a process for preparing an asymmetric compound of Formula I:

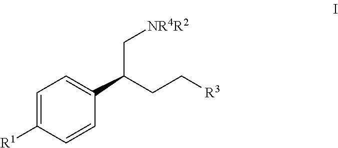

wherein:
R¹ is a leaving group, a halogen, a protected amino group, NO₂, or OH or its protected form;
R² is H;
R³ is —COOR⁵, —CH₂R⁶, or a protected aldehyde; or
R² and R³ are combined to form

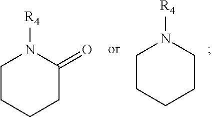

R⁴ is H or an amine protecting group;
R⁵ is C₁₋₆ alkyl, C₃₋₆ cycloalkyl, C₄₋₁₀ heterocyclyl, aryl, or heteroaryl; and, R⁶ is a leaving group or OH or its protected form;
   the process comprising contacting a compound of Formula II:

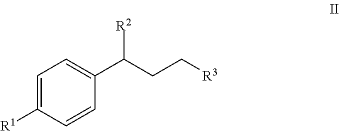

wherein:
R^{1′} is a leaving group, a halogen, a protected amino group, NO₂, or OH or its protected form;
R^{2′} is an aldehyde or an aldehyde equivalent; and
R^{3′} is —COOR⁵, —CH₂R⁶, or a protected aldehyde; or
R^{2′} and R^{3′} are combined to form

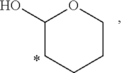

where * represents the point of attachment;
with the engineered transaminase polypeptide of any one of the embodiments disclosed herein in the presence of a coenzyme and an amino donor.

In another aspect, the present invention provides a process for preparing a compound of Formula III, or a salt thereof:

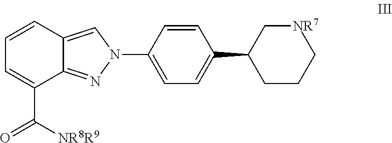

wherein:
R⁷ is H, an amine protecting group, C₁-C₆ alkyl, or tert-butyl;
R⁸ is H;
R⁹ is H, C₁-C₆ alkyl, or tert-butyl; the process comprising contacting a compound of Formula I, or a salt thereof, produced by the process according to any one of the embodiments disclosed herein,

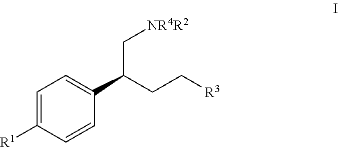

wherein:
R¹ is a halogen;
R² is H;
R³ is —CH₂R⁶;
R⁴ is H or an amine protecting group; and
R⁶ is OH or its protected form;
   with a compound of Formula IV,

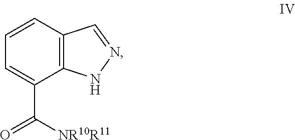

wherein:
R¹⁰ is H;
R¹¹ is C₁-C₆ alkyl or tert-butyl.

In one aspect, the present invention provides a process for preparing niraparib tosylate monohydrate of Formula V:

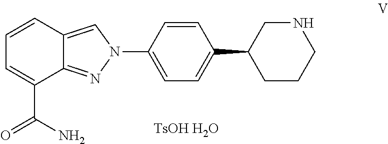

comprising contacting a compound of Formula III, or a salt thereof, produced by the process according to any one of the embodiments disclosed herein, with an acid,

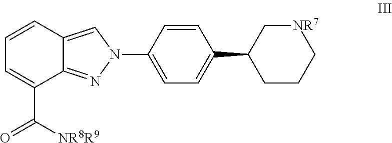

wherein:
R⁷ is H or an amine protecting group;
R⁸ is H;
R⁹ is C₁-C₆ alkyl or tert-butyl.

### DETAILED DESCRIPTION OF THE INVENTION

The present invention provides highly efficient transaminase variants that significantly improves the enzymatic activity and retains high stereoselectivity of the product. Such efficient transaminase variants are achieved by engineering the transaminase variants. The present invention provides transaminase variants. A transaminase variant is an enzyme having transaminase activity and at least one substitution and/or modification relative to the SEQ ID NO: 2. The present invention further provides transaminase variants that comprise multiple (two or more) amino acid substitutions and/or modifications, relative to SEQ ID NO: 2.

In one embodiment there is provided an engineered transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 2.

The present invention provides a transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein X124 is I.

The present invention provides a transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein X124 is I and the polypeptide has an improved enzymatic property over SEQ ID NO: 2.

The present invention provides a transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein X124 is I and the polypeptide has an increased enzymatic activity over SEQ ID NO: 2.

The present invention provides a transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein X124 is I and at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 2, and the polypeptide has an improved enzymatic property over SEQ ID NO: 2.

The present invention provides a transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, X124 is I and the polypeptide has an improved enzymatic property over SEQ ID NO: 2.

The present invention provides a transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, X124 is I and at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 2, and the polypeptide has an improved enzymatic property over SEQ ID NO: 2.

In one embodiment the improved enzymatic property is increased enzymatic activity. In one embodiment, the transaminase enzymes of the present invention exhibit increased activity of at least 1.5-fold improvement over the enzymatic activity of the transaminase enzyme of SEQ ID NO: 2. In one embodiment, the transaminase enzymes of the present invention exhibit increased activity of at least 2-fold, 5-fold, 10-fold, 20-fold, 30-fold, 40-fold, or 50-fold improvement over the enzymatic activity of SEQ ID NO: 2 and have stereoselectivity of greater than 95, 96, 97, 98 or 99% e.e.

In one embodiment there is provided an engineered transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 2, and wherein X124 is I.

In another embodiment, provided herein is an engineered transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises at least one feature selected from the group consisting of: X3 is substituted with H; X5 is substituted with I; X48 is substituted with R; X61 is C, N, or M; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R.

In another embodiment, provided herein an engineered transaminase polypeptide comprising the amino acid sequence of SEQ ID NO: 2 or a functional fragment thereof, wherein the amino acid sequence comprises at least one feature selected from the group consisting of: X3 is substituted with H; X5 is substituted with I; X48 is substituted with R; X61 is C, N, or M; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R; and wherein X124 is I. In another embodiment, provided herein is an engineered transaminase polypeptide comprising the amino acid sequence of SEQ ID NO: 2 or a functional fragment thereof, wherein the amino acid sequence comprises at least one feature selected from the group consisting of: X48 is substituted with R; X61 is C; X69 is C; X94 is substituted with C; X196 is substituted with R; and X297 is substituted with R. In another embodiment, provided herein is an engineered transaminase polypeptide comprising the amino acid sequence of SEQ ID NO: 2 or a functional fragment thereof, wherein the amino acid sequence comprises at least one feature selected from the group consisting of: X48 is substituted with R; X61 is N; X69 is C or T; X94 is substituted with C; X196 is substituted with R; and X297 is substituted with R. In another embodiment, provided herein is an engineered transaminase polypeptide comprising the amino acid sequence of SEQ ID NO: 2 or a functional fragment thereof, wherein the amino acid sequence comprises at least one feature selected from the group consisting of: X48 is substituted with R; X61 is M; X69 is C; X94 is substituted with C; X196 is substituted with R; and X297 is substituted with R. In another embodiment, provided herein is an engineered transaminase polypeptide comprising the amino acid sequence of SEQ ID NO: 2 or a functional fragment thereof, wherein the amino acid sequence comprises at least one feature selected from the group consisting of: X48 is substituted with R; X61 is M; X69 is C; X94 is substituted with C; X137 is substituted with T; X196 is substituted with R; and X297 is substituted with R.

In another embodiment, provided herein is an engineered transaminase polypeptide comprising the amino acid sequence of SEQ ID NO: 2 or a functional fragment thereof, wherein the amino acid sequence comprises X61C, X61N, or X61M substitution. In another embodiment, provided herein is an engineered transaminase polypeptide comprising the amino acid sequence of SEQ ID NO: 2 or a functional fragment thereof, wherein the amino acid sequence comprises X69C or X69T substitution. In another embodiment, provided herein is an engineered transaminase polypeptide comprising the amino acid sequence of SEQ ID NO: 2 or a functional fragment thereof, wherein the amino acid sequence comprises X137T substitution. In another embodiment, provided herein is an engineered transaminase polypeptide comprising the amino acid sequence of SEQ ID NO: 2 or a functional fragment thereof, wherein the amino acid sequence comprises X140Q substitution. In another embodiment, provided herein is an engineered transaminase polypeptide comprising the amino acid sequence of SEQ ID NO: 2 or a functional fragment thereof, wherein the amino acid sequence comprises X199T substitution. In another embodiment, provided herein is an engineered transaminase polypeptide comprising the amino acid sequence of SEQ ID NO: 2 or a functional fragment thereof, wherein the amino acid sequence comprises X202C substitution. In another embodiment, provided herein is an engineered transaminase polypeptide comprising the amino acid sequence of SEQ ID NO: 2 or a functional fragment thereof, wherein the amino acid sequence comprises X3H substitution. In another embodiment, provided herein is an engineered transaminase polypeptide comprising the amino acid sequence of SEQ ID NO: 2 or a functional fragment thereof, wherein the amino acid sequence comprises X5I substitution.

In another embodiment, provided herein is an engineered transaminase polypeptide comprising the amino acid sequence of SEQ ID NO: 2 or a functional fragment thereof, wherein at least two, at least three, at least four, at least five, at least ten, or at least twenty amino acids are substituted to a different amino acid to that found at the corresponding amino acid position in SEQ ID NO: 2. In another embodiment, provided herein is an engineered transaminase polypeptide comprising the amino acid sequence of SEQ ID NO: 2 or a functional fragment thereof, wherein the amino acid sequence comprises X61M and X69T substitutions. In another embodiment, provided herein is an engineered transaminase polypeptide comprising the amino acid sequence of SEQ ID NO: 2 or a functional fragment thereof, wherein the amino acid sequence comprises X61M, X69T, and X137T substitutions. In another embodiment, provided herein is an engineered transaminase polypeptide comprising the amino acid sequence of SEQ ID NO: 2 or a functional fragment thereof, wherein the amino acid sequence comprises X61M, X69T, X137T, X140Q, X199T, and X202C substitutions. In another embodiment, provided herein is an engineered transaminase polypeptide comprising the amino acid sequence of SEQ ID NO: 2 or a functional fragment thereof, wherein the amino acid sequence comprises X3H, X5I, X61M, X69T, X97E, X137T, and X269C substitutions.

In one embodiment, provided herein is an engineered transaminase polypeptide comprising the amino acid sequence of SEQ ID NO: 2 or a functional fragment thereof, wherein the amino acid sequence comprises X61M substitution and at least one other substitution. In another embodiment, provided herein is an engineered transaminase polypeptide comprising the amino acid sequence of SEQ ID NO: 2 or a functional fragment thereof, wherein the amino acid sequence comprises X61M substitution and at least one amino acid substitution selected from the group consisting of: X3 is substituted with H; X5 is substituted with I; X48 is substituted with R; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R.

In another embodiment, provided herein is an engineered transaminase polypeptide comprising the amino acid sequence of SEQ ID NO: 2 or a functional fragment thereof, wherein the amino acid sequence comprises X61M substitution, and at least one other amino acid substitution selected from the group consisting of: X3H, X5I, X69T, X97E, X137T, X140Q, X199T, X202C, and X269C. In another embodiment, provided herein is an engineered transaminase polypeptide comprising the amino acid sequence of SEQ ID NO: 2 or a functional fragment thereof, wherein the amino acid sequence comprises X61M and X3H substitutions. In another embodiment, provided herein is an engineered transaminase polypeptide comprising the amino acid sequence of SEQ ID NO: 2 or a functional fragment thereof, wherein the amino acid sequence comprises X61M and X5I substitutions. In another embodiment, provided herein is an engineered transaminase polypeptide comprising the amino acid sequence of SEQ ID NO: 2 or a functional fragment thereof, wherein the amino acid sequence comprises X61M and X97E substitutions. In another embodiment, provided herein is an engineered transaminase polypeptide comprising the amino acid sequence of SEQ ID NO: 2 or a functional fragment thereof, wherein the amino acid sequence comprises X61M and X137T substitutions. In another embodiment, provided herein is an engineered transaminase polypeptide comprising the amino acid sequence of SEQ ID NO: 2 or a functional fragment thereof, wherein the amino acid sequence comprises X61M and X140Q substitutions. In another embodiment, provided herein is an engineered transaminase polypeptide comprising the amino acid sequence of SEQ ID NO: 2 or a functional fragment thereof, wherein the amino acid sequence comprises X61M and X199T substitutions. In another embodiment, provided herein is an engineered transaminase polypeptide comprising the amino acid sequence of SEQ ID NO: 2 or a functional fragment thereof, wherein the amino acid sequence comprises X61M and X202C substitutions. In another embodiment, provided herein is an engineered transaminase polypeptide comprising the amino acid sequence of SEQ ID NO: 2 or a functional fragment thereof, wherein the amino acid sequence comprises X61M and X269C substitutions.

In another embodiment, provided herein is an engineered transaminase polypeptide comprising the amino acid sequence of SEQ ID NO: 2 or a functional fragment thereof, wherein the amino acid sequence comprises X69T substitution, and at least one other amino acid substitution selected from the group consisting of: X3H, X5I, X61N, X61C, X61M, X97E, X137T, X140Q, X199T, X202C, and X269C. In another embodiment, provided herein is an engineered transaminase polypeptide comprising the amino acid sequence of SEQ ID NO: 2 or a functional fragment thereof, wherein the amino acid sequence comprises X69T and X3H substitutions. In another embodiment, provided herein is an engineered transaminase polypeptide comprising the amino acid sequence of SEQ ID NO: 2 or a functional fragment thereof, wherein the amino acid sequence comprises X69T and X5I substitutions. In another embodiment, provided herein is an engineered transaminase polypeptide comprising the amino acid sequence of SEQ ID NO: 2 or a functional fragment thereof, wherein the amino acid sequence comprises X69T and X97E substitutions. In another embodiment, provided herein is an engineered transaminase polypeptide comprising the amino acid sequence of SEQ ID NO: 2 or a functional fragment thereof, wherein the amino acid sequence comprises X69T and X137T substitutions. In another embodiment, provided herein is an engineered transaminase polypeptide comprising the amino acid sequence of SEQ ID NO: 2 or a functional fragment thereof, wherein the amino acid sequence comprises X69T and X140Q substitutions. In another embodiment, provided herein is an engineered transaminase polypeptide comprising the amino acid sequence of SEQ ID NO: 2 or a functional fragment thereof, wherein the amino acid sequence comprises X69T and X199T substitutions. In another embodiment, provided herein is an engineered transaminase polypeptide comprising the amino acid sequence of SEQ ID NO: 2 or a functional fragment thereof, wherein the amino acid sequence comprises X69T and X202C substitutions. In another embodiment, provided herein is an engineered transaminase polypeptide comprising the amino acid sequence of SEQ ID NO: 2 or a functional fragment thereof, wherein the amino acid sequence comprises X69T and X269C substitutions.

In another embodiment, provided herein is an engineered transaminase polypeptide comprising an amino acid sequence having at least 80%, 85%, 90%, 95%, 99%, or 100% sequence identity to the amino acid sequence set forth in an amino acid sequence selected from the group consisting of SEQ ID NO: 4, SEQ ID NO: 6, SEQ ID NO: 8, SEQ ID NO: 10, SEQ ID NO: 14, SEQ ID NO: 16, SEQ ID NO: 18, and SEQ ID NO: 20. One of ordinary skill in the art would appreciate that the initial M of SEQ ID NOs: 4, 6, 8, 10, 14, 16, 18, or 20 can be modified or deleted without affecting the function of the engineered transaminase polypeptide. In another embodiment, the engineered transaminase polypeptide comprises an N-terminal tag having the formula M-Hₓ (SEQ ID NO: 21), wherein M is a methionine residue and Hₓ represents a chain of x histidine residues, where x is a whole integer between 0 and 20. In certain embodiments, x is 0, 1, 2, 3, 4, 5, 6, 7, 8, 9, 10, 15 or 20.

In further aspect, there is provided an engineered transaminase polypeptide comprising an amino acid sequence having at least 80%, 85%, 90%, 95%, 96%, 97%, 98%, or 99% sequence identity to the amino acid sequence set forth in SEQ ID NO: 4 or a functional fragment thereof, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 4.

In one embodiment there is provided an engineered transaminase polypeptide comprising an amino acid sequence having at least 80%, 85%, 90%, 95%, 96%, 97%, 98%, or 99% sequence identity to the amino acid sequence set forth in SEQ ID NO: 4 or a functional fragment thereof, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 4, and wherein X124 is I.

In another embodiment, provided herein is an engineered transaminase polypeptide comprising an amino acid sequence having at least 80%, 85%, 90%, 95%, 96%, 97%, 98%, or 99% sequence identity to the amino acid sequence set forth in SEQ ID NO: 4 or a functional fragment thereof, wherein the amino acid sequence comprises at least one feature selected from the group consisting of: X3 is substituted with H; X5 is substituted with I; X48 is substituted with R; X61 is N or M; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R.

In another embodiment, provided herein an engineered transaminase polypeptide comprising the amino acid sequence of SEQ ID NO: 4 or a functional fragment thereof, wherein the amino acid sequence comprises at least one feature selected from the group consisting of: X3 is substituted with H; X5 is substituted with I; X48 is substituted with R; X61 is N or M; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R; and wherein X124 is I. In another embodiment, provided herein is an engineered transaminase polypeptide comprising the amino acid sequence of SEQ ID NO: 4 or a functional fragment thereof, wherein the amino acid sequence comprises at least one feature selected from the group consisting of: X48 is substituted with R; X61 is N; X69 is C or T; X94 is substituted with C; X196 is substituted with R; and X297 is substituted with R. In another embodiment, provided herein is an engineered transaminase polypeptide comprising the amino acid sequence of SEQ ID NO: 4 or a functional fragment thereof, wherein the amino acid sequence comprises at least one feature selected from the group consisting of: X48 is substituted with R; X61 is M; X69 is C; X94 is substituted with C; X196 is substituted with R; and X297 is substituted with R. In another embodiment, provided herein is an engineered transaminase polypeptide comprising the amino acid sequence of SEQ ID NO: 4 or a functional fragment thereof, wherein the amino acid sequence comprises at least one feature selected from the group consisting of: X48 is substituted with R; X61 is M; X69 is C; X94 is substituted with C; X137 is substituted with T; X196 is substituted with R; and X297 is substituted with R.

In another embodiment, provided herein is an engineered transaminase polypeptide comprising the amino acid sequence of SEQ ID NO: 4 or a functional fragment thereof, wherein the amino acid sequence comprises X61N or X61M substitution. In another embodiment, provided herein is an engineered transaminase polypeptide comprising the amino acid sequence of SEQ ID NO: 4 or a functional fragment thereof, wherein the amino acid sequence comprises X69N or X69T substitution. In another embodiment, provided herein is an engineered transaminase polypeptide comprising the amino acid sequence of SEQ ID NO: 4 or a functional fragment thereof, wherein the amino acid sequence comprises X137T substitution. In another embodiment, provided herein is an engineered transaminase polypeptide comprising the amino acid sequence of SEQ ID NO: 4 or a functional fragment thereof, wherein the amino acid sequence comprises X140Q substitution. In another embodiment, provided herein is an engineered transaminase polypeptide comprising the amino acid sequence of SEQ ID NO: 4 or a functional fragment thereof, wherein the amino acid sequence comprises X199T substitution. In another embodiment, provided herein is an engineered transaminase polypeptide comprising the amino acid sequence of SEQ ID NO: 4 or a functional fragment thereof, wherein the amino acid sequence comprises X202C substitution. In another embodiment, provided herein is an engineered transaminase polypeptide comprising the amino acid sequence of SEQ ID NO: 4 or a functional fragment thereof, wherein the amino acid sequence comprises X3H substitution. In another embodiment, provided herein is an engineered transaminase polypeptide comprising the amino acid sequence of SEQ ID NO: 4 or a functional fragment thereof, wherein the amino acid sequence comprises X5I substitution.

In another embodiment, provided herein is an engineered transaminase polypeptide comprising the amino acid sequence of SEQ ID NO: 4 or a functional fragment thereof, wherein at least two, at least three, at least four, at least five, at least ten, or at least twenty amino acids are substituted to a different amino acid to that found at the corresponding amino acid position in SEQ ID NO: 4. In another embodiment, provided herein is an engineered transaminase polypeptide comprising the amino acid sequence of SEQ ID NO: 4 or a functional fragment thereof, wherein the amino acid sequence comprises X61M and X69T substitutions. In another embodiment, provided herein is an engineered transaminase polypeptide comprising the amino acid sequence of SEQ ID NO: 4 or a functional fragment thereof, wherein the amino acid sequence comprises X61M, X69T, and X137T substitutions. In another embodiment, provided herein is an engineered transaminase polypeptide comprising the amino acid sequence of SEQ ID NO: 4 or a functional fragment thereof, wherein the amino acid sequence comprises X61M, X69T, X137T, X140Q, X199T, and X202C substitutions. In another embodiment, provided herein is an engineered transaminase polypeptide comprising the amino acid sequence of SEQ ID NO: 4 or a functional fragment thereof, wherein the amino acid sequence comprises X3H, X5I, X61M, X69T, X97E, X137T, and X269C substitutions.

In one embodiment, provided herein is an engineered transaminase polypeptide comprising the amino acid sequence of SEQ ID NO: 4 or a functional fragment thereof, wherein the amino acid sequence comprises X61M substitution and at least one other substitution. In another embodiment, provided herein is an engineered transaminase polypeptide comprising the amino acid sequence of SEQ ID NO: 4 or a functional fragment thereof, wherein the amino acid sequence comprises X61M substitution and at least one amino acid substitution selected from the group consisting of: X3 is substituted with H; X5 is substituted with I; X48 is substituted with R; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R.

In another embodiment, provided herein is an engineered transaminase polypeptide comprising the amino acid sequence of SEQ ID NO: 4 or a functional fragment thereof, wherein the amino acid sequence comprises X61M substitution, and at least one other amino acid substitution selected from the group consisting of: X3H, X5I, X69T, X97E, X137T, X140Q, X199T, X202C, and X269C. In another embodiment, provided herein is an engineered transaminase polypeptide comprising the amino acid sequence of SEQ ID NO: 4 or a functional fragment thereof, wherein the amino acid sequence comprises X61M and X3H substitutions. In another embodiment, provided herein is an engineered transaminase polypeptide comprising the amino acid sequence of SEQ ID NO: 4 or a functional fragment thereof, wherein the amino acid sequence comprises X61M and X5I substitutions. In another embodiment, provided herein is an engineered transaminase polypeptide comprising the amino acid sequence of SEQ ID NO: 4 or a functional fragment thereof, wherein the amino acid sequence comprises X61M and X97E substitutions. In another embodiment, provided herein is an engineered transaminase polypeptide comprising the amino acid sequence of SEQ ID NO: 4 or a functional fragment thereof, wherein the amino acid sequence comprises X61M and X137T substitutions. In another embodiment, provided herein is an engineered transaminase polypeptide comprising the amino acid sequence of SEQ ID NO: 4 or a functional fragment thereof, wherein the amino acid sequence comprises X61M and X140Q substitutions. In another embodiment, provided herein is an engineered transaminase polypeptide comprising the amino acid sequence of SEQ ID NO: 4 or a functional fragment thereof, wherein the amino acid sequence comprises X61M and X199T substitutions. In another embodiment, provided herein is an engineered transaminase polypeptide comprising the amino acid sequence of SEQ ID NO: 4 or a functional fragment thereof, wherein the amino acid sequence comprises X61M and X202C substitutions. In another embodiment, provided herein is an engineered transaminase polypeptide comprising the amino acid sequence of SEQ ID NO: 4 or a functional fragment thereof, wherein the amino acid sequence comprises X61M and X269C substitutions.

In another embodiment, provided herein is an engineered transaminase polypeptide comprising the amino acid sequence of SEQ ID NO: 4 or a functional fragment thereof, wherein the amino acid sequence comprises X69T substitution, and at least one other amino acid substitution selected from the group consisting of: X3H, X5I, X61N, X61C, X61M, X97E, X137T, X140Q, X199T, X202C, and X269C. In another embodiment, provided herein is an engineered transaminase polypeptide comprising the amino acid sequence of SEQ ID NO: 4 or a functional fragment thereof, wherein the amino acid sequence comprises X69T and X3H substitutions. In another embodiment, provided herein is an engineered transaminase polypeptide comprising the amino acid sequence of SEQ ID NO: 4 or a functional fragment thereof, wherein the amino acid sequence comprises X69T and X5I substitutions. In another embodiment, provided herein is an engineered transaminase polypeptide comprising the amino acid sequence of SEQ ID NO: 4 or a functional fragment thereof, wherein the amino acid sequence comprises X69T and X97E substitutions. In another embodiment, provided herein is an engineered transaminase polypeptide comprising the amino acid sequence of SEQ ID NO: 4 or a functional fragment thereof, wherein the amino acid sequence comprises X69T and X137T substitutions. In another embodiment, provided herein is an engineered transaminase polypeptide comprising the amino acid sequence of SEQ ID NO: 4 or a functional fragment thereof, wherein the amino acid sequence comprises X69T and X140Q substitutions. In another embodiment, provided herein is an engineered transaminase polypeptide comprising the amino acid sequence of SEQ ID NO: 4 or a functional fragment thereof, wherein the amino acid sequence comprises X69T and X199T substitutions. In another embodiment, provided herein is an engineered transaminase polypeptide comprising the amino acid sequence of SEQ ID NO: 4 or a functional fragment thereof, wherein the amino acid sequence comprises X69T and X202C substitutions. In another embodiment, provided herein is an engineered transaminase polypeptide comprising the amino acid sequence of SEQ ID NO: 4 or a functional fragment thereof, wherein the amino acid sequence comprises X69T and X269C substitutions.

In some embodiments, a single point mutation in the engineered transaminase polypeptide can increase the enzymatic activity by between about 1.1-fold and about 10-fold, by between about 1.2-fold and about 7-fold, by between about 1.3-fold and about 5-fold, by between about 1.5-fold and about 2.5-fold or by between about 1.5-fold and about 6-fold or by at least 2-fold compared to the activity of the engineered transaminase of SEQ ID NO: 2. The positive effect of single point mutations can multiply when combined as multiple point mutations in an engineered transaminase polypeptide. In some embodiments, the engineered transaminase polypeptide can contain at least 2, 3, 4, 5, 6, 7, 8, 9, 10, 11, 12, 13, 14, 15, 16, 17, 18, 19 or 20 amino acid substitutions. In some embodiments, while the activity of the engineered transaminase increases, the stereoselectivity remains over 95%, 96%, 97%, 98%, or 99% e.e. In some embodiments, use of an engineered transaminase polypeptide disclosed herein increases the transaminase activity by at least about 2-fold, at least about 5-fold, at least about 10-fold, at least about 15-fold, at least about 20-fold, at least about 25-fold, at least about 30-fold, at least about 35-fold, at least about 40-fold, at least about 45-fold, at least about 50-fold, or at least about 55-fold compared to the activity of the engineered transaminase of SEQ ID NO: 2. In some embodiments, use of an engineered transaminase polypeptide disclosed herein increases the transaminase activity by at least about 2 times, at least about 5 times, at least about 10 times, at least about 15 times, at least about 20 times, at least about 25 times, at least about 30 times, at least about 35 times, at least about 40 times, at least about 45 times, at least about 50 times, or at least about 55 times compared to the activity of the engineered transaminase of SEQ ID NO: 2. In some embodiments, the control transaminase is an engineered transaminase comprising the amino acid sequence of SEQ ID NO: 2.

In one embodiment there is provided an engineered transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises the feature that: at least one amino acid selected from the group consisting of amino acid F3, H5, P48, C61, V69, I94, I97, E137, I140, I196, I199, T202, P269, and S297 is substituted to a different amino acid to that found at the corresponding amino acid position in SEQ ID NO: 2, and wherein X124 is I.

In some embodiments, the engineered transaminase polypeptide of any one of preceding embodiments has the feature: X124 is I.

In another embodiment, provided herein is an engineered transaminase polypeptide comprising the amino acid sequence set forth in an amino acid sequence selected from the group consisting of SEQ ID NO: 4, SEQ ID NO: 6, SEQ ID NO: 8, SEQ ID NO: 10, SEQ ID NO: 14, SEQ ID NO: 16, SEQ ID NO: 18, and SEQ ID NO: 20. One of ordinary skill in the art would appreciate that the initial M of SEQ ID NOs: 4, 6, 8, 10, 14, 16, 18, or 20 can be modified or deleted without affecting the function of the engineered transaminase polypeptide.

### Kits

In some embodiments, provided herein is a composition comprising any one of engineered transaminase polypeptides as described herein.

In some embodiments, provided herein is a kit comprising an engineered transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 2, and wherein X124 is I; a coenzyme; and an amino donor. In some embodiments, coenzyme is pyridoxal-phosphate (PLP). In some embodiments, the amino donor is isopropylamine.

In some embodiments, provided herein is a kit comprising an engineered transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises at least one feature selected from the group consisting of: X3 is substituted with H; X5 is substituted with I; X48 is substituted with R; X61 is C, N, or M; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R; a coenzyme; and an amino donor. In some embodiments, coenzyme is pyridoxal-phosphate (PLP). In some embodiments, the amino donor is isopropylamine.

In some embodiments, provided herein is a kit comprising an engineered transaminase polypeptide comprising the amino acid sequence of SEQ ID NO: 2 or a functional fragment thereof, wherein the amino acid sequence comprises X69T substitution, and at least one other amino acid substitution selected from the group consisting of: X3H, X5I, X61N, X61C, X61M, X97E, X137T, X140Q, X199T, X202C, and X269C; a coenzyme; and an amino donor.

In some embodiments, provided herein is a kit comprising an engineered transaminase polypeptide comprising an amino acid sequence having at least 80%, 85%, 90%, 95%, 96%, 97%, 98%, or 99% sequence identity to the amino acid sequence set forth in SEQ ID NO: 4 or a functional fragment thereof, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 4, and wherein X124 is I; a coenzyme; and an amino donor.

In some embodiments, provided herein is a kit comprising an engineered transaminase polypeptide comprising an amino acid sequence having at least 80%, 85%, 90%, 95%, 96%, 97%, 98%, or 99% sequence identity to the amino acid sequence set forth in SEQ ID NO: 4 or a functional fragment thereof, wherein the amino acid sequence comprises at least one feature selected from the group consisting of: X3 is substituted with H; X5 is substituted with I; X48 is substituted with R; X61 is N or M; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R; a coenzyme; and an amino donor.

In some embodiments, provided herein is a kit comprising an engineered transaminase polypeptide, a coenzyme, and an amino donor as described herein.

### Nucleic Acids

In another aspect, provided herein are polynucleotides encoding the engineered transaminase polypeptide of the present invention. In some embodiments, the polynucleotide encodes an engineered transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 2, and wherein X124 is I.

In some embodiments, the polynucleotide encodes an engineered transaminase polypeptide comprising the amino acid sequence of SEQ ID NO: 2 or a functional fragment thereof, wherein the amino acid sequence comprises at least one feature selected from the group consisting of: X3 is substituted with H; X5 is substituted with I; X48 is substituted with R; X61 is C, N, or M; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R; and wherein X124 is I.

In some embodiments, the polynucleotide encodes an engineered transaminase polypeptide comprising the amino acid sequence of SEQ ID NO: 2 or a functional fragment thereof, wherein the amino acid sequence comprises X61M substitution and at least one amino acid substitution selected from the group consisting of: X3 is substituted with H; X5 is substituted with I; X48 is substituted with R; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R.

In some embodiments, the polynucleotide encodes an engineered transaminase polypeptide comprising the amino acid sequence of SEQ ID NO: 2 or a functional fragment thereof, wherein the amino acid sequence comprises X61M substitution, and at least one other amino acid substitution selected from the group consisting of: X3H, X5I, X69T, X97E, X137T, X140Q, X199T, X202C, and X269C.

In some embodiments, the polynucleotide encodes an engineered transaminase polypeptide comprising the amino acid sequence of SEQ ID NO: 2 or a functional fragment thereof, wherein the amino acid sequence comprises X69T substitution, and at least one other amino acid substitution selected from the group consisting of: X3H, X5I, X61N, X61C, X61M, X97E, X137T, X140Q, X199T, X202C, and X269C.

In some embodiments, the polynucleotide encodes an engineered transaminase polypeptide comprising the amino acid sequence of SEQ ID NO: 2 or a functional fragment thereof, wherein the amino acid sequence comprises X61M and X69T substitutions.

In some embodiments, the polynucleotide encodes an engineered transaminase polypeptide comprising the amino acid sequence of SEQ ID NO: 2 or a functional fragment thereof, wherein at least two, at least three, at least four, at least five, at least ten, or at least twenty amino acids are substituted to a different amino acid to that found at the corresponding amino acid position in SEQ ID NO: 2.

In some embodiments, the polynucleotide comprises a polynucleotide sequence having at least 80%, 85%, 90%, 95%, 99%, or 100% sequence identity to the polynucleotide sequence set forth in a polynucleotide sequence selected from the group consisting of SEQ ID NO: 3, SEQ ID NO: 5, SEQ ID NO: 7, SEQ ID NO: 9, SEQ ID NO: 13, SEQ ID NO: 15, SEQ ID NO: 17, or SEQ ID NO: 19.

In some embodiments, provided herein is a polynucleotide that encodes the engineered transaminase polypeptide of any one of preceding embodiments as described herein.

In some embodiments, provided herein is a vector comprising the polynucleotides encoding the engineered transaminase polypeptide of the present invention. In some embodiments, the vector comprises a polynucleotide that encodes an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 2, and wherein X124 is I.

In some embodiments, provided herein is a vector that comprises a polynucleotide that encodes an engineered transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises at least one feature selected from the group consisting of: X3 is substituted with H; X5 is substituted with I; X48 is substituted with R; X61 is C, N, or M; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R.

In some embodiments, provided herein is a vector that comprises a polynucleotide that encodes an engineered transaminase polypeptide comprising the amino acid sequence of SEQ ID NO: 2 or a functional fragment thereof, wherein the amino acid sequence comprises X61M substitution and at least one amino acid substitution selected from the group consisting of: X3 is substituted with H; X5 is substituted with I; X48 is substituted with R; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R.

In some embodiments, provided herein is a vector that comprises a polynucleotide that encodes an engineered transaminase polypeptide comprising the amino acid sequence of SEQ ID NO: 2 or a functional fragment thereof, wherein the amino acid sequence comprises X61M substitution, and at least one other amino acid substitution selected from the group consisting of: X3H, X5I, X69T, X97E, X137T, X140Q, X199T, X202C, and X269C.

In some embodiments, provided herein is a vector that comprises a polynucleotide that encodes an engineered transaminase polypeptide comprising the amino acid sequence of SEQ ID NO: 2 or a functional fragment thereof, wherein the amino acid sequence comprises X69T substitution, and at least one other amino acid substitution selected from the group consisting of: X3H, X5I, X61N, X61C, X61M, X97E, X137T, X140Q, X199T, X202C, and X269C.

In some embodiments, provided herein is a vector that comprises a polynucleotide that encodes the engineered transaminase polypeptide of any one of preceding embodiments as described herein.

### Host Cells

In another aspect, provided herein is a host cell comprising an engineered transaminase polypeptide, wherein the engineered transaminase polypeptide comprises at least one amino acid substitution. In another embodiment, the host cell is E. coli. In some embodiments, the host cell comprises two or more engineered transaminase polypeptides.

In another aspect, provided herein is a host cell comprising a polynucleotide provided herein (e.g., encoding the engineered transaminase polypeptides provided herein). In some embodiments, the host cell comprises two or more polynucleotides provided herein (e.g., 2, 3, 4, 5, 6, 7, 8, 9, 10 or more nucleic acids).

In some embodiments, provided herein is a host cell comprising a vector comprising the polynucleotide that encode the engineered transaminase polypeptides of the present invention. In some embodiments, the host cell comprises a vector which comprises a polynucleotide that encodes an engineered transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 2, and wherein X124 is I.

In some embodiments, provided herein is a host cell comprising a vector which comprises a polynucleotide that encodes an engineered transaminase polypeptide comprising an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises at least one feature selected from the group consisting of: X3 is substituted with H; X5 is substituted with I; X48 is substituted with R; X61 is C, N, or M; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R.

In some embodiments, provided herein is a host cell comprising a vector which comprises a polynucleotide that encodes an engineered transaminase polypeptide comprising the amino acid sequence of SEQ ID NO: 2 or a functional fragment thereof, wherein the amino acid sequence comprises X61M substitution and at least one amino acid substitution selected from the group consisting of: X3 is substituted with H; X5 is substituted with I; X48 is substituted with R; X69 is C or T; X94 is substituted with C; X97 is substituted with E; X137 is substituted with T; X140 is substituted with Q; X196 is substituted with R; X199 is substituted with T; X202 is substituted with C; X269 is substituted with C or V; and X297 is substituted with R.

In some embodiments, provided herein is a host cell comprising a vector which comprises a polynucleotide that encodes an engineered transaminase polypeptide comprising the amino acid sequence of SEQ ID NO: 2 or a functional fragment thereof, wherein the amino acid sequence comprises X61M substitution, and at least one other amino acid substitution selected from the group consisting of: X3H, X5I, X69T, X97E, X137T, X140Q, X199T, X202C, and X269C.

In some embodiments, provided herein is a host cell comprising a vector which comprises a polynucleotide that encodes an engineered transaminase polypeptide comprising the amino acid sequence of SEQ ID NO: 2 or a functional fragment thereof, wherein the amino acid sequence comprises X69T substitution, and at least one other amino acid substitution selected from the group consisting of: X3H, X5I, X61N, X61C, X61M, X97E, X137T, X140Q, X199T, X202C, and X269C.

In some embodiments, provided herein is a host cell comprising a vector that comprises a polynucleotide that encodes engineered transaminase polypeptide of any one of preceding embodiments as described herein.

### Synthetic Process

In one aspect, the present invention provides an efficient process for the preparation of an asymmetric compound of Formula I:

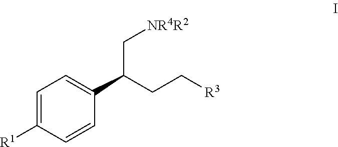

wherein:
R¹ is a leaving group, a halogen, a protected amino group, —NO₂, or —OH or its protected form;
R² is H;
R³ is —COOR⁵, —CH₂R⁶, or a protected aldehyde; or
R² and R³ are combined to form

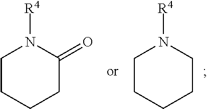

R⁴ is H or an amine protecting group;
R⁵ is C₁₋₆ alkyl, C₃₋₁₀ cycloalkyl, C₄₋₁₀ heterocyclyl, aryl, or heteroaryl; and,
R⁶ is a leaving group or —OH or its protected form.

In one embodiment, R¹ is a leaving group. In one embodiment, R¹ is halogen. In one embodiment, R¹ is Br.

In another embodiment, R² and R³ are combined to form

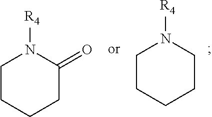

wherein R⁴ is H. In a further embodiment, R² and R³ are combined to form

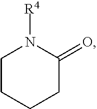

wherein R⁴ is H.

In one embodiment, R¹ is Br, R² and R³ are combined to form

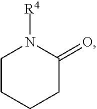

wherein R⁴ is H.

In another embodiment, R² is H, R³ is CH₂R⁶, R⁴ is H, and R⁶ is OH.

In a further aspect, the present invention provides a process for the preparation of an asymmetric compound of Formula I, comprising contacting any one of engineered transaminase polypeptides of the present invention with a compound of Formula II:

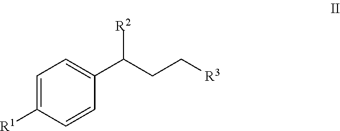

wherein:
R^{1′} is a leaving group, a halogen, a protected amino group, —NO₂, or —OH or its protected form;
R^{2′} is an aldehyde or an aldehyde equivalent; and,
R^{3′} is —COOR⁵, —CH₂R⁶, or a protected aldehyde; or
R^{2′} and R^{3′} are combined to form

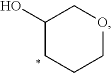

where * represents the point of attachment.

In one embodiment, R^{1′} is a leaving group. In one embodiment, R^{1′} is halogen. In one embodiment, R^{1′} is Br.

In one embodiment, R^{2′} and R^{3′} are combined to form

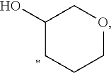

where * represents the point of attachment.

Thus, in one embodiment, there is provided a process for the preparation of an asymmetric compound of Formula I:

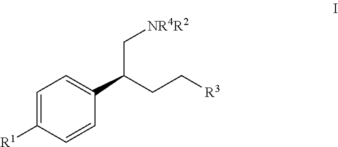

wherein:
R¹ is a halogen;
R² is H;
R³ is —CH₂R⁶;
R⁴ is H or an amine protecting group; and
R⁶ is OH or its protected form;
   the process comprising contacting a compound of Formula IIa:

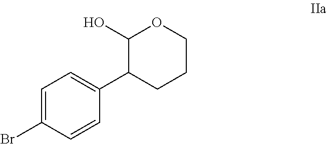 [SMILES: OC1OCCCC1c1ccc(Br)cc1]

with the engineered transaminase polypeptide of any one of preceding embodiments disclosed herein in the presence of a coenzyme and an amino donor to provide a compound of Formula Ia:

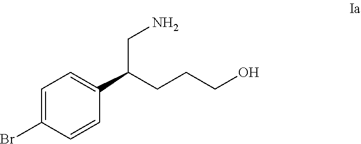 [SMILES: NC[C@@H](CCCO)c1ccc(Br)cc1]

the process optionally comprising the step of protecting the NH₂ and OH groups of Formula Ia to provide a compound of Formula Ib:

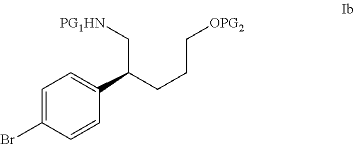

wherein PG₁ is an amine protecting group and wherein PG₂ is an oxygen protecting group. In one embodiment, the process according to any one of the embodiments disclosed herein can also be a process for the preparation of niraparib, including niraparib tosylate monohydrate.

In one embodiment, there is provided a process for the preparation of an asymmetric compound of Formula I:

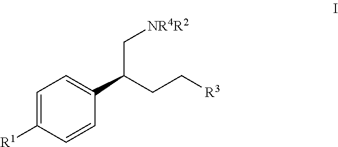

wherein:
R¹ is Br;
R² is H;
R³ is —CH₂R⁶;
R⁴ is H or an amine protecting group; and
R⁶ is OH or its protected form;
   the process comprising contacting a compound of Formula IIa:

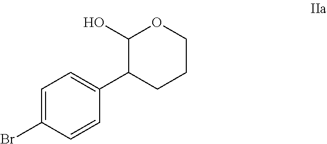 [SMILES: OC1OCCCC1c1ccc(Br)cc1]

with the engineered transaminase polypeptide of any one of embodiments disclosed herein in the presence of a coenzyme and an amino donor to provide a compound of Formula Ia:

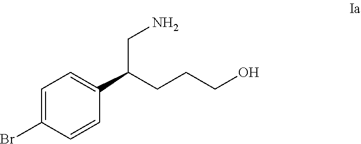 [SMILES: NC[C@@H](CCCO)c1ccc(Br)cc1]

In some embodiments, the process optionally comprising the step of protecting the NH₂ group of Formula Ia to provide a compound of Formula Ic:

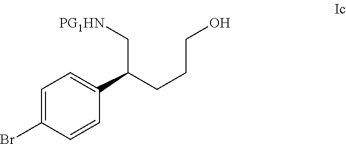

wherein PG₁ is an amine protecting group. This optional step applies when R⁴ is an amine protecting group.

In some embodiments, the process further optionally comprising the step of protecting the OH group of Formula Ic to provide a compound of Formula Ib:

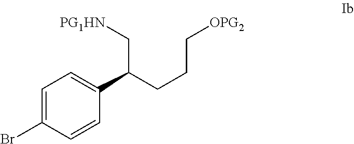

wherein PG₂ is a hydroxyl protecting group. This optional step applies when R⁶ is OH in its protected form and R⁴ is an amine protecting group.

In one embodiment, amine protecting group is selected from the group consisting of: formyl, acetyl (Ac), trifluoroacetyl, benzyl (Bn), benzoyl (Bz), carbamate, benzyloxycarbonyl (“CBZ”), p-methoxybenzyl carbonyl (Moz or MeOZ), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”), p-methoxybenzyl (PMB), tosyl (Ts), 3,4-dimethoxybenzyl (DMPM), p-methoxyphenyl (PMP), 2-naphthylmethyl ether (Nap), and trichloroethyl chloroformate (Troc). In one embodiment, amine protecting group is tert-butoxycarbonyl (“Boc”).

In one embodiment, hydroxyl protecting group is selected from the group consisting of: methyl esters, ethyl esters, acetate, propionate groups, glycol esters, benzyl, trityl ethers, alkyl ethers, tetrahydropyranyl ethers, trialkylsilyl ethers, TMS, TIPS, mesylate, and allyl ethers. In one embodiment, hydroxyl protecting group is mesylate.

In one embodiment, the compound of Formula II is selected from the group consisting of:

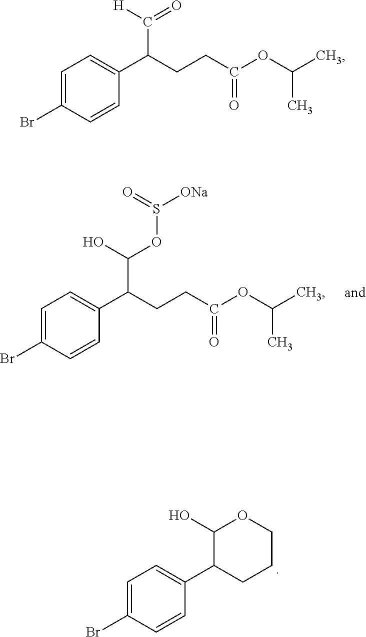

In one embodiment, the compound of Formula I is selected from the group consisting of:

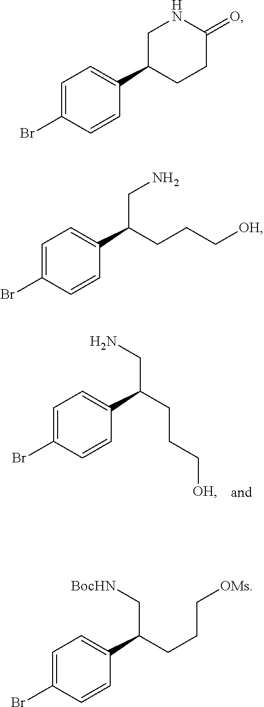 [SMILES: O=C1CC[C@@H](c2ccc(Br)cc2)CN1 ; NC[C@@H](CCCO)c1ccc(Br)cc1 ; NC[C@@H](CCCO)c1ccc(Br)cc1 ; CC(C)(C)OC(=O)NC[C@@H](CCCOS(C)(=O)=O)c1ccc(Br)cc1]

In one embodiment, the present invention provides a process for the preparation of an asymmetric compound,

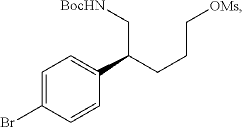 [SMILES: CC(C)(C)OC(=O)NC[C@@H](CCCOS(C)(=O)=O)c1ccc(Br)cc1]

comprising contacting any one of engineered transaminase polypeptides of the present invention with

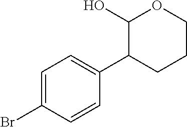 [SMILES: OC1OCCCC1c1ccc(Br)cc1]

in the presence of a coenzyme and an amino donor.

In one embodiment, the present invention provides a process for the preparation of an asymmetric compound,

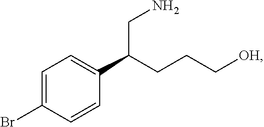 [SMILES: NC[C@@H](CCCO)c1ccc(Br)cc1]

comprising contacting any one of engineered transaminase polypeptides of the present invention with

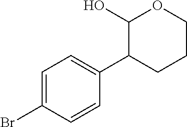 [SMILES: OC1OCCCC1c1ccc(Br)cc1]

in the presence of a coenzyme and an amino donor.

In one embodiment, the present invention provides a process for the preparation of an asymmetric compound,

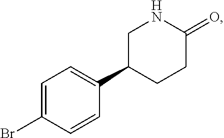 [SMILES: O=C1CC[C@@H](c2ccc(Br)cc2)CN1]

comprising contacting any one of engineered transaminase polypeptides of the present invention with

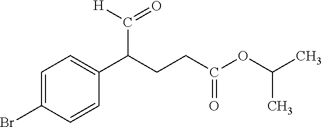 [SMILES: CC(C)OC(=O)CCC(C=O)c1ccc(Br)cc1]

in the presence of a coenzyme and an amino donor.

In one embodiment, the present invention provides a process for the preparation of an asymmetric compound,

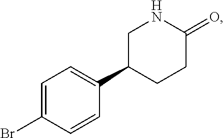 [SMILES: O=C1CC[C@@H](c2ccc(Br)cc2)CN1]

comprising contacting any one of engineered transaminase polypeptides of the present invention with

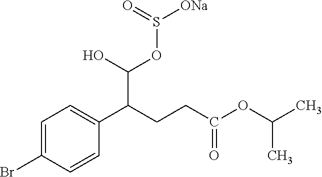

in the presence of a coenzyme and an amino donor.

In one embodiment, the process of any preceding embodiments provides a compound of Formula I having an enantiomeric excess (e.e.) of at least about 95% e.e., at least about 96% e.e., at least about 97% e.e., at least about 98% e.e., or at least about 99.9% e.e. In another embodiment, the transaminase-catalyzed process of a compound of Formula II as described provides a compound of Formula I having an enantiomeric excess of at least 95%. In a further embodiment, the transaminase-catalyzed process of a compound of Formula II as described provides a compound of Formula I having an enantiomeric excess of at least 99%. In one embodiment, the compound of Formula I is selected from the group consisting of:

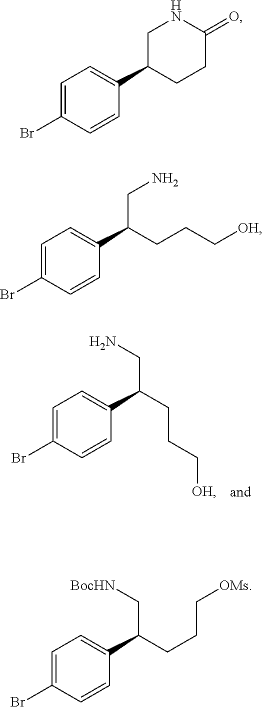 [SMILES: O=C1CC[C@@H](c2ccc(Br)cc2)CN1 ; NC[C@@H](CCCO)c1ccc(Br)cc1 ; NC[C@@H](CCCO)c1ccc(Br)cc1 ; CC(C)(C)OC(=O)NC[C@@H](CCCOS(C)(=O)=O)c1ccc(Br)cc1]

In one embodiment, the compound of Formula I is selected from the group consisting of:

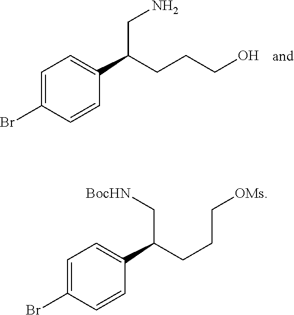 [SMILES: NC[C@@H](CCCO)c1ccc(Br)cc1 ; CC(C)(C)OC(=O)NC[C@@H](CCCOS(C)(=O)=O)c1ccc(Br)cc1]

In one embodiment, the compound of Formula I is

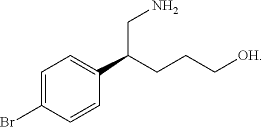 [SMILES: NC[C@@H](CCCO)c1ccc(Br)cc1]

In one embodiment, the compound of Formula I is

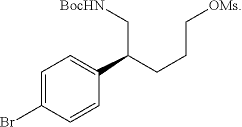 [SMILES: CC(C)(C)OC(=O)NC[C@@H](CCCOS(C)(=O)=O)c1ccc(Br)cc1]

In one embodiment of the process of any preceding processes disclosed herein, the engineered transaminase polypeptide comprises an amino acid sequence having at least 80%, 85%, 90%, 95%, 96%, 97%, 98%, or 99% sequence identity to the amino acid sequence set forth in SEQ ID NO: 2 or a functional fragment thereof, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 2. In some embodiments, the engineered transaminase polypeptide is selected from the group consisting of the amino acid sequence as set forth in SEQ ID NOs: 4, 6, 8, 10, 14, 16, 18, and 20. In one embodiment, the engineered transaminase polypeptide is selected from the group consisting of the amino acid sequence as set forth in SEQ ID NOs: 16, 18, and 20. In some embodiments, the engineered transaminase polypeptide comprises any one of the engineered transaminase polypeptides of preceding embodiments.

In one embodiment of the process of any preceding processes disclosed herein, the coenzyme is pyridoxal-phosphate (PLP). In some embodiments, the amino donor is isopropylamine.

In one aspect, the present invention provides a process for preparing a compound of Formula III, or a salt thereof:

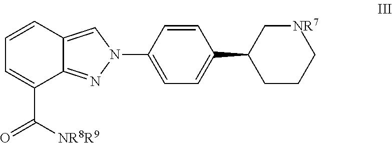

wherein:
R⁷ is H, an amine protecting group, alkyl, C₁-C₄ alkyl, or tert-butyl;
R⁸ is H;
R⁹ is H, alkyl, C₁-C₄ alkyl, or tert-butyl; the process comprising contacting a compound of Formula I, or a salt thereof, produced by the process according to any one of the embodiments disclosed herein,

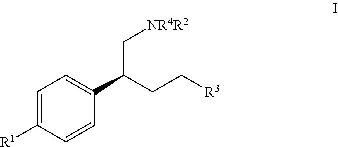

wherein:
R¹ is halogen;
R² is H;
R³ is —CH₂R⁶;
R⁴ is H or an amine protecting group; and
R⁶ is OH or its protected form;
   with a compound of Formula IV,

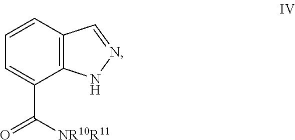

wherein:
R¹⁰ is H;
R¹¹ is alkyl, C₁-C₄ alkyl or tert-butyl.

In one embodiment, R⁷ is an amine protecting group. In one embodiment, the amine protecting group comprises tert-butyloxycarbonyl (Boc), 9-fluorenylmethyloxycarbonyl (Fmoc), carboxybenzyl group (Cbz), p-methoxybenzyl carbonyl (Moz), acetyl (Ac), benzoyl (Bz), p-methoxybenzyl (PMB), 3,4-dimethoxybenzyl (DMPM), p-methoxyphenyl (PMP), 2-naphthylmethyl ether (Nap), tosyl (Ts), or trichloroethyl chloroformate (Troc). In one embodiment, the amine protecting group is tert-butyloxycarbonyl group (Boc).

In one embodiment, R⁹ is tert-butyl (^{t}Bu). In another embodiment, R¹¹ is tert-butyl (^{t}Bu).

In one embodiment, the step of contacting a compound of Formula I, or a salt thereof is performed in presence of

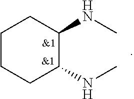

In another embodiment, the step of contacting is in presence of at least one agent selected from the group consisting of: toluene, potassium tert-butoxide, tripotassium phosphate (K₃PO₄), copper(I) bromide, ammonium hydroxide, and heptane, or any combination thereof. In yet another embodiment, the step of contacting is in presence of a solvent. Consistent with these embodiments, the solvent comprises, but is not limited to, toluene, N,N-dimethylformide (DMF), t-butanol, dimethoxyethane (DME), acetonitrile, dichloromethane (DCM), tetrahydrofuran (THF), 2-methyltetrahydrofuran (ME-THF), isopropyl alcohol, methanol, ethanol, or any combination thereof. In some embodiments, the solvent comprises toluene.

In one embodiment, the step of contacting is performed in presence of an acid. In one embodiment, the acid comprises formic acid, acetic acid, propionic acid, butyric acid, valeric acid, caproic acid, oxalic acid, lactic acid, malic acid, citric acid, benzoic acid, carbonic acid, uric acid, taurine, p-toluenesulfonic acid, trifluoromethanesulfonic acid, aminomethylphosphonic acid, trifluoroacetic acid (TFA), phosphonic acid, sulfuric acid, nitric acid, phosphoric acid, hydrochloric acid, ethane sulfonic acid (ESA), methanesulfonic acid (MsOH) or any combination thereof. In another embodiment, the acid is methanesulfonic acid (MsOH).

In one embodiment, the compound of Formula I, or salt thereof, has a structure of:

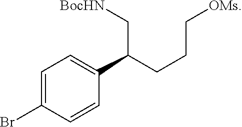 [SMILES: CC(C)(C)OC(=O)NC[C@@H](CCCOS(C)(=O)=O)c1ccc(Br)cc1]

In one embodiment, the compound of Formula IV, or salt thereof, has a structure of:

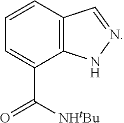

In one embodiment, the compound of Formula III, or salt thereof, has a structure of:

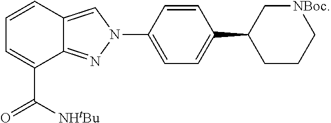

In one aspect, the present invention provides a process for preparing niraparib tosylate monohydrate of Formula V:

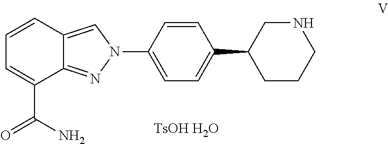

comprising contacting a compound of Formula III, or a salt thereof, produced by the process according to any one of the embodiments disclosed herein, with an acid,

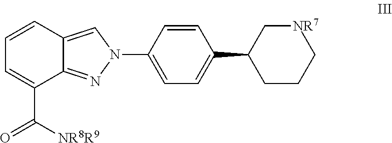

wherein:
R⁷ is H or an amine protecting group;
R⁸ is H;
R⁹ is C₁-C₄ alkyl or tert-butyl.

In one embodiment, R⁷ is an amine protecting group. In one embodiment, the amine protecting group is tert-butyloxycarbonyl (Boc), 9-fluorenylmethyloxycarbonyl (Fmoc), carboxybenzyl group (Cbz), p-methoxybenzyl carbonyl (Moz), acetyl (Ac), benzoyl (Bz), p-methoxybenzyl (PMB), 3,4-dimethoxybenzyl (DMPM), p-methoxyphenyl (PMP), 2-naphthylmethyl ether (Nap), tosyl (Ts), or trichloroethyl chloroformate (Troc). In one embodiment, the amine protecting group is tert-butyloxycarbonyl group (Boc). In one embodiment, R⁹ is tert-butyl.

In one embodiment, the acid is at least one acid selected from the group consisting of: methanesulfonic acid (MsOH), formic acid, acetic acid, propionic acid, butyric acid, valeric acid, caproic acid, oxalic acid, lactic acid, malic acid, citric acid, benzoic acid, carbonic acid, uric acid, taurine, p-toluenesulfonic acid, trifluoromethanesulfonic acid, aminomethylphosphonic acid, trifluoroacetic acid (TFA), phosphonic acid, sulfuric acid, nitric acid, phosphoric acid, hydrochloric acid, and ethane sulfonic acid (ESA), or any combination thereof. In one embodiment, the acid is methanesulfonic acid (MsOH). In one embodiment, the acid is p-toluenesulfonic acid.

In one embodiment, the step of contacting is in presence of a solvent. In one embodiment, the solvent comprises N,N-dimethylformide (DMF), t-butanol, dimethoxyethane (DME), acetonitrile, dichloromethane (DCM), tetrahydrofuran (THF), 2-methyltetrahydrofuran (ME-TIF), isopropyl alcohol, methanol, ethanol, or any combination thereof. In another embodiment, the solvent comprises ME-THF.

In one embodiment, the process of any one of preceding embodiments, further comprising the process of preparing a compound of Formula V,

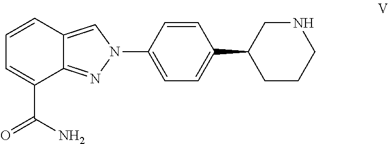 [SMILES: NC(=O)c1cccc2cn(-c3ccc([C@@H]4CCCNC4)cc3)nc12]

wherein the process comprises contacting a compound of Formula III with an acid. In one embodiment, wherein contacting a compound of Formula III with an acid additionally provides a compound of Formula VI,

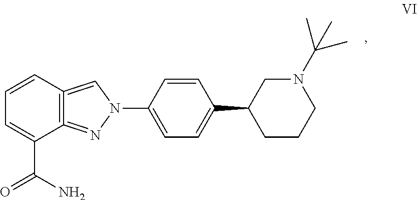

wherein R⁹ is tert-butyl.

In one embodiment, there is provided a process for preparing niraparib tosylate monohydrate of Formula VII, comprising:

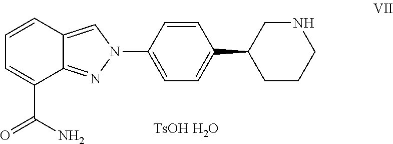

contacting a compound of Formula V, or a salt thereof, produced by the process according to any one of preceding embodiments, with a methanesulfonic acid or a para-toluenesulfonic acid,

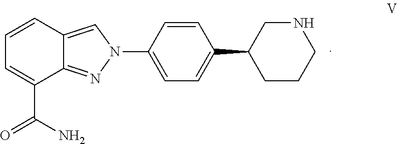 [SMILES: NC(=O)c1cccc2cn(-c3ccc([C@@H]4CCCNC4)cc3)nc12]

In one embodiment, wherein the contacting is in presence of a solvent. In one embodiment, the solvent is water.

In one aspect, the present invention provides a composition, comprising
i) niraparib tosylate monohydrate of Formula VII:

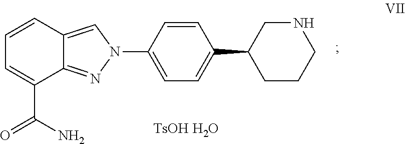

and
ii) less than 0.1 weight % of the compound of Formula VI, or a salt thereof,

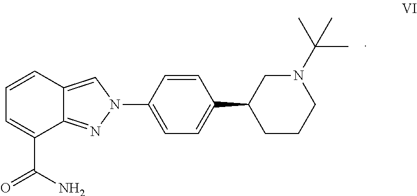 [SMILES: CC(C)(C)N1CCC[C@@H](c2ccc(-n3cc4cccc(C(N)=O)c4n3)cc2)C1]

In one embodiment, the concentration of the compound of Formula VI is present in the composition less than 0.09 weight %, less than 0.08 weight %, less than 0.07 weight %, less than 0.06 weight %, less than 0.05 weight %, less than 0.04 weight %, less than 0.03 weight %, less than 0.02 or less than 0.01 weight %.

In one embodiment, the compound of Formula VI is present after contacting a compound of Formula III with an acid according to the process for preparing niraparib tosylate monohydrate of Formula V disclosed herein.

In one embodiment, the acid is a strong acid. In one embodiment, the acid is at least one acid selected from the group consisting of: methanesulfonic acid (MsOH), formic acid, acetic acid, propionic acid, butyric acid, valeric acid, caproic acid, oxalic acid, lactic acid, malic acid, citric acid, benzoic acid, carbonic acid, uric acid, taurine, p-toluenesulfonic acid, trifluoromethanesulfonic acid, aminomethylphosphonic acid, trifluoroacetic acid (TFA), phosphonic acid, sulfuric acid, nitric acid, phosphoric acid, hydrochloric acid, and ethane sulfonic acid (ESA), or any combination thereof. In one embodiment, the acid is methanesulfonic acid (MsOH).

In some embodiments, the niraparib tosylate monohydrate is added to a solvent. In some embodiments, the niraparib tosylate monohydrate is added to a solvent to form a crystalline form of the niraparib tosylate monohydrate. In some embodiment, the solvent is dimethyl sulfoxide (DMSO). In some embodiments, the process according to any one of preceding embodiments, further comprising wet milling the niraparib tosylate monohydrate disclosed herein. In some embodiments, the process further comprising annealing the niraparib tosylate monohydrate using one or more temperature cycles.

In some embodiments, the solvent comprises DMSO. In some embodiments, the process comprises contacting the niraparib tosylate monohydrate with DMSO and water. In some embodiments, a water to solvent ratio (v/v) of from about 200:1 to about 1:200 is used in the contacting. In some embodiments, the water to solvent ratio (v/v) is from about 200:1 to about 1:200, for example, from about 200:1 to about 100:1, from about 200:1 to about 10:1, from about 200:1 to about 5:1, from about 200:1 to about 2:1, from about 200:1 to about 1:1, from about 200:1 to about 1:2, from about 200:1 to about 1:5, from about 200:1 to about 1:10, from about 200:1 to about 1:100, from about 100:1 to about 10:1, from about 100:1 to about 5:1, from about 100:1 to about 2:1, from about 100:1 to about 1:1, from about 100:1 to about 1:2, from about 100:1 to about 1:5, from about 100:1 to about 1:10, from about 100:1 to about 1:100, from about 100:1 to about 1:200, from about 10:1 to about 5:1, from about 10:1 to about 2:1, from about 10:1 to about 1:1, from about 10:1 to about 1:2, from about 10:1 to about 1:5, from about 10:1 to about 1:10, from about 10:1 to about 1:100, from about 10:1 to about 1:200, from about 5:1 to about 2:1, from about 5:1 to about 1:1, from about 5:1 to about 1:2, from about 5:1 to about 1:5, from about 5:1 to about 1:10, from about 5:1 to about 1:100, from about 5:1 to about 1:200, from about 2:1 to about 1:1, from about 2:1 to about 1:2, from about 2:1 to about 1:5, from about 2:1 to about 1:10, from about 2:1 to about 1:100, from about 2:1 to about 1:200, from about 1:1 to about 1:2, from about 1:1 to about 1:5, from about 1:1 to about 1:10, from about 1:1 to about 1:100, from about 1:1 to about 1:200, from about 1:2 to about 1:5, from about 1:2 to about 1:10, from about 1:2 to about 1:100, from about 1:2 to about 1:200, from about 1:5 to about 1:10, from about 1:5 to about 1:100, from about 1:5 to about 1:200, from about 1:10 to about 1:100, from about 1:10 to about 1:200, or from about 1:100 to about 1:200. In some embodiments, the water to solvent ratio (v/v) is from about 5:1 to about 1:5.

In one aspect, use of the engineered transaminase polypeptide of any one of preceding embodiments in preparing an asymmetric compound of Formula I:

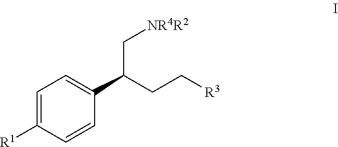

wherein:
R¹ is a halogen, a protected amino group, —NO₂, or —OH or its protected form;
R² is H;
R³ is —COOR⁵, —CH₂R⁶, or a protected aldehyde; or,
R² and R³ are combined to form

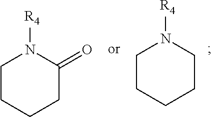

R⁴ is H or an amine protecting group;
R⁵ is C₁₋₆ alkyl, C₃₋₆ cycloalkyl, C₄₋₁₀ heterocyclyl, aryl, or heteroaryl; and,
R⁶ is a leaving group or —OH or its protected form is disclosed.

In one embodiment according to the use of preceding embodiments, the engineered transaminase polypeptide is selected from the group consisting of the amino acid sequence as set forth in SEQ ID NOs: 4, 6, 8, 10, 14, 16, 18, and 20. In some embodiments, the engineered transaminase polypeptide is selected from the group consisting of the amino acid sequence as set forth in SEQ ID NOs: 16, 18, and 20.

In one embodiment according to the use of any one of preceding embodiments, R² and R³ are combined to form

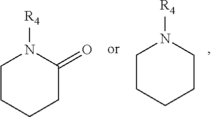

and R⁴ is H. In one embodiment, R² and R³ are combined to form

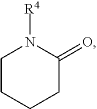

and R⁴ is H. In some embodiments, R¹ is Br, R² is H, R³ is CH₂R⁶, R⁴ is H, and R⁶ is OH.

### Definitions

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. The term “at least one” refers to one or more.

Additionally, numerical limitations given with respect to concentrations or levels of a substance, such as solution component concentrations or ratios thereof, and reaction conditions such as temperatures, pressures, and cycle times are intended to be approximate. Unless specified otherwise, where a numerical range is provided, it is inclusive, i.e., the endpoints are included.

For the purposes of the descriptions herein, the abbreviations used for the genetically encoded amino acids are conventional and are as follows in Table 1:

TABLE 1
Abbreviations

| AMINO ACID | THREE-LETTER | ONE-LETTER |
|---|---|---|
| ALANINE | ALA | A |
| ARGININE | ARG | R |
| ASPARAGINE | ASN | N |
| ASPARTATE | ASP | D |
| CYSTEINE | CYS | C |
| GLUTAMATE | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

When either one-letter or three-letter abbreviations are used, unless specifically preceded by an “L” or a “D” or clear from the context in which the abbreviation is used, the amino acid may be in either the L- or D-configuration about alpha-carbon (C-alpha). For example, whereas “Ala” designates alanine without specifying the configuration about the alpha-carbon, “D-Ala” and “L-Ala” designate D-alanine and L-alanine, respectively. When peptide sequences are presented as a string of one-letter or three-letter abbreviations, the sequences are presented in the N→C direction in accordance with convention.

“About” or “approximately” mean roughly, around, or in the regions of. The terms “about” or “approximately” further mean within an acceptable contextual error range for the particular value as determined by one of ordinary skill in the art, which will depend in part on how the value is measured, i.e., the limitations of the measurement system or the degree of precision required for a particular purpose. When the terms “about” or “approximately” are used in conjunction with a numerical range, it modifies that range by extending the boundaries above and below the numerical values set forth.

“Acidic amino acid or residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Amino acid” or “residue” as used in the context of the polypeptides disclosed herein refers to the specific monomer at a sequence position (e.g., P5 indicates that the “amino acid” or “residue” at position 5 is a proline).

“Amino acid difference” or “residue difference” or “amino acid substitution” refers to a change in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X61 as compared to SEQ ID NO: 2” refers to a change of the amino acid residue at the polypeptide position corresponding to position 61 of SEQ ID NO: 2. Thus, if the reference polypeptide of SEQ ID NO: 2 has a tyrosine at position 61, then a “residue difference at position X61 as compared to SEQ ID NO: 2” is an amino acid substitution of any residue other than tyrosine at the position of the polypeptide corresponding to position 61 of SEQ ID NO: 2. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some instances (e.g., Tables 7 and 8), the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, a polypeptide of the present invention can include one or more amino acid residue differences relative to a reference sequence, which is indicated by a list of the specified positions where changes are made relative to the reference sequence. The present invention includes engineered polypeptide sequences comprising one or more amino acid differences that include either/or both conservative and non-conservative amino acid substitutions.

“Amino acceptor” and “amine acceptor,” “keto substrate,” “keto,” and “ketone” are used interchangeably herein to refer to a carbonyl (keto, or ketone) compound which accepts an amino group from a donor amine. Amino acceptors are molecules of general formula shown below,

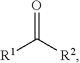

in which each of R¹, R², when taken independently, is an alkyl, an alkylaryl group, or aryl group which is unsubstituted or substituted with one or more enzymatically acceptable groups. R¹ may be the same or different from R² in structure or chirality. In some embodiments, R¹ and R², taken together, may form a ring that is unsubstituted, substituted, or fused to other rings. Amino acceptors include keto carboxylic acids and alkanones (ketones). Typical keto carboxylic acids are a-keto carboxylic acids such as glyoxalic acid, pyruvic acid, oxaloacetic acid, and the like, as well as salts of these acids. Amino acceptors also include substances which are converted to an amino acceptor by other enzymes or whole cell processes, such as fumaric acid (which can be converted to oxaloacetic acid), glucose (which can be converted to pyruvate), lactate, maleic acid, and others. Amino acceptors that can be used include, by way of example and not limitation, (R)-2-(3,4-dimethoxyphenethoxy)cyclohexanone, 3,4-dihydronaphthalen-1 (2H)-one, 1-phenylbutan-2-one, 3,3-dimethylbutan-2-one, octan-2-one, ethyl 3-oxobutanoate, 4-phenylbutan-2-one, 1-(4-bromophenyl)ethanone, 2-methyl-cyclohexamone, 7-methoxy-2-tetralone, 1-hydroxybutan-2-one, pyruvic acid, acetophenone, (R)-2-(3,4-dimethoxyphenethoxy)cyclohexanone, 2-methoxy-5-fluoroacetophenone, levulinic acid, 1-phenylpropan-1-one, 1-(4-bromophenyl)propan-1-one, 1-(4-nitrophenyl)propan-1-one, 1-phenylpropan-2-one, 2-oxo-3-methylbutanoic acid, 1-(3-trifluoromethylphenyl)propan-1-one, hydroxypropanone, methoxyoxypropanone, 1-phenylbutan-1-one, 1-(2,5-dimethoxy-4-methylphenyl)butan-2-one, 1-(4-hydroxyphenyl)butan-3-one, 2-acetylnaphthalene, phenylpyruvic acid, 2-ketoglutaric acid, and 2-ketosuccinic acid, including both (R) and (S) single isomers where possible.

“Amino donor” or “amine donor” refers to an amino compound which donates an amino group to the amino acceptor, thereby becoming a carbonyl species. Amino donors are molecules of general formula shown below,

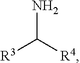

in which each of R³, R⁴, when taken independently, is an alkyl, an alkylaryl group, or aryl group which is unsubstituted or substituted with one or more enzymatically non-inhibiting groups. R³ can be the same or different from R⁴ in structure or chirality. In some embodiments, R³ and R⁴, taken together, may form a ring that is unsubstituted, substituted, or fused to other rings. Typical amino donors that can be used with the embodiments of the present invention include chiral and achiral amino acids, and chiral and achiral amines. Amino donors that can be used with the embodiments herein include, by way of example and not limitation, isopropylamine (also referred to as 2-aminopropane, and referred to elsewhere herein as “PM”), a-phenethylamine (also termed 1-phenylethanamine), and its enantiomers (S)-1-phenylethanamine and (R)-1-phenylethanamine, 2-amino-4-phenylbutane, glycine, L-glutamic acid, L-glutamate, monosodium glutamate, L-alanine, D-alanine, D,L-alanine, L-aspartic acid, L-lysine, D,L-ornithine, β-alanine, taurine, n-octylamine, cyclohexylamine, 1,4-butanediamine (also referred to as putrescine), 1,6-hexanediamine, 6-aminohexanoic acid, 4-aminobutyric acid, tyramine, and benzyl amine, 2-aminobutane, 2-amino-1-butanol, 1-amino-1-phenylethane, 1-amino-1-(2-methoxy-5-fluorophenyl)ethane, 1-amino-1-phenylpropane, 1-amino-1-(4-hydroxyphenyl)propane, 1-amino-1-(4-bromophenyl)propane, 1-amino-1-(4-nitrophenyl)propane, l-phenyl-2-aminopropane, 1-(3-trifluoromethylphenyl)-2-aminopropane, 2-aminopropanol, 1-amino-1-phenylbutane, 1-phenyl-2-aminobutane, 1-(2,5-dimethoxy-4-methylphenyl)-2-aminobutane, 1-phenyl-3-aminobutane, 1-(4-hydroxyphenyl)-3-aminobutane, 1-amino-2-methylcyclopentane, 1-amino-3-methylcyclopentane, 1-amino-2-methylcyclohexane, 1-amino-1-(2-naphthyl)ethane, 3-methylcyclopentylamine, 2-methylcyclopentylamine, 2-ethylcyclopentylamine, 2-methylcyclohexylamine, 3-methylcyclohexylamine, 1-aminotetralin, 2-aminotetralin, 2-amino-5-methoxytetralin, and 1-aminoindan, including both (R) and (S) single isomers where possible and including all possible salts of the amines.

“Aliphatic amino acid or residue” refers to a hydrophobic amino acid or residue having an aliphatic hydrocarbon side chain. Genetically encoded aliphatic amino acids include L-Ala (A), L-Val (V), L-Leu (L), and L-Ile (I).

“And/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

“Aromatic amino acid or residue” refers to a hydrophilic or hydrophobic amino acid or residue having a side chain that includes at least one aromatic or heteroaromatic ring. Genetically encoded aromatic amino acids include L-Phe (F), L-Tyr (Y) and L-Trp (W). Although owing to the pKa of its heteroaromatic nitrogen atom L-His (H) it is sometimes classified as a basic residue, or as an aromatic residue as its side chain includes a heteroaromatic ring, herein histidine is classified as a hydrophilic residue or as a “constrained residue” (see below).

“Basic amino acid or residue” refers to a hydrophilic amino acid or residue having a side chain exhibiting a pKa value of greater than about 6 when the amino acid is included in a peptide or polypeptide. Basic amino acids typically have positively charged side chains at physiological pH due to association with hydronium ions. Genetically encoded basic amino acids include L-Arg (R) and L-Lys (K).

“Chiral amine” refers to amines of general formula R¹—CH(NH₂)—R² and is employed herein in its broadest sense, including a wide variety of aliphatic and alicyclic compounds of different, and mixed, functional types, characterized by the presence of a primary amino group bound to a secondary carbon atom which, in addition to a hydrogen atom (H), carries either (i) a divalent group forming a chiral cyclic structure, or (ii) two substituents (other than hydrogen) differing from each other in structure or chirality. Divalent groups forming a chiral cyclic structure include, for example, 2-methylbutane-1,4-diyl, pentane-1,4-diyl, hexane-1,4-diyl, hexane-1,5-diyl, 2-methylpentane-1,5-diyl. The two different substituents on the secondary carbon atom (R¹ and R² above) also can vary widely and include alkyl, aralkyl, aryl, halo, hydroxy, lower alkyl, lower alkoxy, lower alkylthio, cycloalkyl, carboxy, carbalkoxy, carbamoyl, mono- and di-(lower alkyl) substituted carbamoyl, trifluoromethyl, phenyl, nitro, amino, mono- and di-(lower alkyl) substituted amino, alkylsulfonyl, arylsulfonyl, alkylcarboxamido, arylcarboxamido, etc., as well as alkyl, aralkyl, or aryl substituted by the foregoing.

“Cofactor,” as used herein, refers to a non-protein compound that operates in combination with an enzyme in catalyzing a reaction. “Pyridoxal-phosphate,” “PLP,” “pyridoxal-5′-phosphate,” “PYP,” and “P5P” are used interchangeably herein to refer to the compound that acts as a cofactor in transaminase reactions. In some embodiments, pyridoxal phosphate is defined by the structure 1-(4′-formyl-3′-hydroxy-2′-methyl-5′-pyridyl)methoxyphosphonic acid, CAS number [54-47-7], Pyridoxal-5′-phosphate can be produced in vivo by phosphorylation and oxidation of pyridoxol (also known as Vitamin B₆). In transamination reactions using transaminase enzymes, the amine group of the amino donor is transferred to the cofactor to produce a keto byproduct, while pyridoxal-5′-phosphate is converted to pyridoxamine phosphate. Pyridoxal-5′-phosphate is regenerated by reaction with a different keto compound (the amino acceptor). The transfer of the amine group from pyridoxamine phosphate to the amino acceptor produces a chiral amine and regenerates the cofactor. In some embodiments, the pyridoxal-5′-phosphate can be replaced by other members of the vitamin B₆ family, including pyridoxine (PN), pyridoxal (PL), pyridoxamine (PM), and their phosphorylated counterparts; pyridoxine phosphate (PNP), and pyridoxamine phosphate (PMP). As used herein, “cofactor” is intended to encompass the vitamin B₆ family compounds PLP, PN, PL, PM, PNP, and PMP, which are sometimes also referred to as coenzymes.

“Coding sequence” refers to that portion of a nucleic acid (e.g., a gene) that encodes an amino acid sequence of a protein.

“Conservative” amino acid substitutions or mutations refer to the interchangeability of residues having similar side chains, and thus typically involves the substitution of the amino acid in the polypeptide with amino acids within the same or similar defined class of amino acids. However, as used herein, in some embodiments, conservative mutations do not include substitutions from a hydrophilic to hydrophilic, hydrophobic to hydrophobic, hydroxyl-containing to hydroxyl-containing, or small to small residue, if the conservative mutation can instead be a substitution from an aliphatic to an aliphatic, non-polar to non-polar, polar to polar, acidic to acidic, basic to basic, aromatic to aromatic, or constrained to constrained residue. Further, as used herein, A, V, L, or I can be conservatively mutated to either another aliphatic residue or to another non-polar residue. Table 2 below shows exemplary conservative substitutions.

TABLE 2
Conservative Substitutions

| Residue | Possible Conservative Mutations |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| P | none |
| N, Q, S, T | Other polar |
| H, Y, W, F | Other aromatic (H, Y, W, F) |
| C | None |

“Constrained amino acid or residue” refers to an amino acid or residue that has a constrained geometry. Herein, constrained residues include L-Pro (P) and L-His (H). Histidine has a constrained geometry because it has a relatively small imidazole ring. Proline has a constrained geometry, because it also has a five-membered ring.

“Conversion” refers to the enzymatic conversion of the substrate(s) to the corresponding product(s). “Percent conversion” refers to the percent of the substrate that is converted to the product within a period of time under specified conditions. Thus, the “enzymatic activity” or “activity” of a transaminase polypeptide can be expressed as “percent conversion” of the substrate to the product.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. Methods of comparing a sequence with a specified reference sequence are known to a skilled person. For example, the Needleman Wunsch method can be used to compare any amino acid or polynucleotide sequence with a reference sequence.

“Corresponding amino acid position” is a term that is widely used and well-understood by a skilled person. A corresponding amino acid position can be identified by aligning the amino acid sequences using any of the well-known amino acid alignment methods. For example, the NCBI BLAST algorithm method can be used to identify a corresponding amino acid position. “Cysteine” or L-Cys (C) is unusual in that it can form disulfide bridges with other L-Cys (C) amino acids or other sulfanyl- or sulfhydryl-containing amino acids. The “cysteine-like residues” include cysteine and other amino acids that contain sulfhydryl moieties that are available for formation of disulfide bridges. The ability of L-Cys (C) (and other amino acids with —SH containing side chains) to exist in a peptide in either the reduced free —SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present invention L-Cys (C) is categorized into its own unique group.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered transaminase enzyme. Deletions can be directed to the internal portions and/or terminal portions of the polypeptide. In various embodiments, the deletion can comprise a continuous segment or can be discontinuous.

“Derived from” as used herein in the context of engineered transaminase enzymes, identifies the originating transaminase enzyme, and/or the gene encoding such transaminase enzyme, upon which the engineering was based.

“Fragment” as used herein, refers to a polypeptide that has an amino-terminal and/or carboxy-terminal deletion, but where the remaining amino acid sequence is identical to the corresponding positions in the sequence. Fragments can be at least 14 amino acids long, at least 20 amino acids long, at least 50 amino acids long or longer, and up to 70%, 80%, 90%, 95%, 98%, and 99%, or more, of the full-length transaminase. A “functional fragment” or a “biologically active fragment”, used interchangeably, herein refers to a polypeptide that has an amino-terminal and/or carboxy-terminal deletion(s) and/or internal deletions, but where the remaining amino acid sequence is identical to the corresponding positions in the sequence to which it is being compared and that retains substantially all of the activity of the full-length polypeptide.

“Improved enzyme property” refers to a transaminase polypeptide that exhibits an improvement in any enzyme property as compared to a reference transaminase. For the engineered transaminase polypeptides described herein, the comparison is generally made to the wild-type transaminase enzyme, although in some embodiments, the reference transaminase can be another improved engineered transaminase. Enzyme properties for which improvement is desirable include, but are not limited to, enzymatic activity (which can be expressed in terms of percent conversion of the substrate), thermo stability, solvent stability, pH activity profile, cofactor requirements, refractoriness to inhibitors (e.g., substrate or product inhibition), stereospecificity, and stereoselectivity (including enantioselectivity). In one embodiment, the transaminase enzymes of the present invention have stereoselectivity of greater than 95, 96, 97, 98 or 99% e.e.

“Increased enzymatic activity” refers to an improved property of the engineered transaminase polypeptides, which can be represented by an increase in specific activity (e.g., product produced/time/weight protein) or an increase in percent conversion of the substrate to the product (e.g., percent conversion of starting amount of substrate to product in a specified time period using a specified amount of transaminase) as compared to the reference transaminase enzyme. Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.1 times the enzymatic activity of the corresponding transaminase enzyme set forth in SEQ ID NO: 2, to as much as 2 times, 5 times, 10 times, 20 times, 25 times, 50 times, 75 times, 100 times, or more enzymatic activity than the naturally occurring transaminase or another engineered transaminase from which the transaminase polypeptides were derived. In specific embodiments, the engineered transaminase enzyme exhibits improved enzymatic activity in the range of 2.0 to 50 times, 2.0 to 100 times greater than that of the control transaminase enzyme having the SEQ ID NO: 2. It is understood by the skilled artisan that the activity of any enzyme is diffusion limited such that the catalytic turnover rate cannot exceed the diffusion rate of the substrate, including any required cofactors. The theoretical maximum of the diffusion limit, or k_{cat}/Kₘ, is generally about 10⁸ to 10⁹ (M s″¹). Hence, any improvements in the enzyme activity of the transaminase will have an upper limit related to the diffusion rate of the substrates acted on by the transaminase enzyme. Transaminase activity can be measured by any one of standard assays, such as by monitoring changes in spectrophotometric properties of reactants or products. Comparisons of enzyme activities are made using a defined preparation of enzyme, a defined assay under a set condition, and one or more defined substrates, as further described in detail herein.

In one embodiment, the transaminase enzymes of the present invention exhibit increased activity of at least 2-fold improvement over the enzymatic activity of SEQ ID NO: 2. In one embodiment, the transaminase enzymes of the present invention exhibit increased activity of at least at least 2-fold, 5-fold, 10-fold, 20-fold, 30-fold, 40-fold, or 50-fold improvement over the enzymatic activity of SEQ ID NO: 2 and have stereoselectivity of greater than 95, 96, 97, 98 or 99% e.e.

“Insertion” refers to modification to the polypeptide by addition of one or more amino acids from the reference polypeptide. In some embodiments, the improved engineered transaminase enzymes comprise insertions of one or more amino acids to the naturally occurring transaminase polypeptide as well as insertions of one or more amino acids to other improved transaminase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Naturally-occurring” or “wild-type” refers to the form found in nature. For example, a naturally occurring or wild-type polypeptide or polynucleotide sequence is a sequence present in an organism that can be isolated from a source in nature and that has not been intentionally modified by human manipulation.

“Non-naturally occurring”, “recombinant” or “engineered” or when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Nucleic acid” herein means a polymeric form of nucleotides of any length, which contain deoxyribonucleotides, ribonucleotides, and/or their analogs. It includes DNA, RNA and DNA/RNA hybrids. It also includes DNA or RNA analogs, such as those containing modified backbones (e.g., peptide nucleic acids (PNAs) or phosphorothioates) or modified bases. Thus, the nucleic acid of the invention includes mRNA, DNA, cDNA, recombinant nucleic acids, branched nucleic acids, plasmids, vectors, etc. Where the nucleic acid takes the form of RNA, it may or may not have a 5′ cap. RNA may be a small, medium, or large RNA. The number of nucleotides per strand of a small RNA is from 10-30 (e.g., siRNAs). A medium RNA contains between 30-2000 nucleotides per strand (e.g., non-self-replicating mRNAs). A large RNA contains at least 2,000 nucleotides per strand e.g., at least 2,500, at least 3,000, at least 4,000, at least 5,000, at least 6,000, at least 7,000, at least 8,000, at least 9,000, or at least 10,000 nucleotides per strand. The molecular mass of a single-stranded RNA molecule in g/mol (or Dalton) can be approximated using the formula: molecular mass=(number of RNA nucleotides)×340 g/mol. RNA can include, in addition to any 5′ cap structure, one or more nucleotides having a modified nucleobase. For instance, an RNA can include one or more modified pyrimidine nucleobases, such as pseudouridine and/or 5 methylcytosine residues. In some embodiments, however, the RNA includes no modified nucleobases, and may include no modified nucleotides i.e., all of the nucleotides in the RNA are standard A, C, G and U ribonucleotides (except for any 5′ cap structure, which may include a 7′ methylguanosine). In other embodiments, the RNA may include a 5′ cap comprising a 7′ methylguanosine, and the first 1, 2 or 3 5′ ribonucleotides may be methylated at the 2′ position of the ribose.

Nucleic acids can be in recombinant form, i.e., a form that does not occur in nature. For example, the nucleic acid may comprise one or more heterologous nucleic acid sequences (e.g., a sequence encoding another antigen and/or a control sequence such as a promoter or an internal ribosome entry site). The nucleic acid may be part of a vector i.e., part of a nucleic acid designed for transduction/transfection of one or more cell types. Vectors may be, for example, “expression vectors,” which are designed for expression of a nucleotide sequence in a host cell, or “viral vectors,” which are designed to result in the production of a recombinant virus or virus-like particle.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see, e.g., Altschul, et al., 1990, J. Mol. Biol. 215: 403-410 and Altschul, et al., 1977, Nucleic Acids Res. 3389-3402). Software for performing BLAST analyses is publicly available through the National Center for Biotechnology Information website.

Numerous other algorithms are available that function similarly to BLAST in providing percent identity for two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel, et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided. The ClustalW program is also suitable for determining identity.

“Protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

“Purification” or “purifying” herein means the process of removing components from a composition or host cell or culture, the presence of which is not desired. Purification is a relative term and does not require that all traces of the undesirable component be removed from the composition. Purification may include processes such as centrifugation, dialyzation, ion-exchange chromatography, and size-exclusion chromatography, affinity-purification, or precipitation. Thus, the term “purified” does not require absolute purity; rather, it is intended as a relative term. A preparation of substantially pure nucleic acid or protein can be purified such that the desired nucleic acid, or protein, represents at least 50% of the total nucleic acid content of the preparation. In certain embodiments, a substantially pure nucleic acid, or protein, will represent at least 60%, at least 70%, at least 80%, at least 85%, at least 90%, or at least 95% or more of the total nucleic acid or protein content of the preparation. Immunogenic molecules or antigens or antibodies which have not been subjected to any purification steps (i.e., the molecule as it is found in nature) are not suitable for pharmaceutical (e.g., vaccine) use.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For example, a “reference sequence based on SEQ ID NO: 2 having at the residue corresponding to X137 is a threonine” refers to a reference sequence in which the corresponding residue at X137 in SEQ ID NO: 2, which is a glutamate (E), has been changed to threonine (T). “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

“Reference to”, “corresponding to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered transaminase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Substantially pure polypeptide” refers to a composition in which the polypeptide species is the predominant species present (i.e., on a molar or weight basis it is more abundant than any other individual macromolecular species in the composition) and is generally a substantially purified composition when the object species comprises at least about 50 percent of the macromolecular species present by mole or % weight. Generally, a substantially pure transaminase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated improved transaminases polypeptide is a substantially pure polypeptide composition.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (e.e.) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (d.e.). Where a mixture contains more than two diastereomers it is common to report the ratio of diastereomers or “diastereomeric ratio” rather than diastereomeric excess. Enantiomeric excess and diastereomeric excess are types of stereomeric excess. “Highly stereoselective” refers to a transaminase polypeptide that is capable of converting the substrate to the corresponding chiral amine product with at least about 90%, 95%, or 99% stereomeric excess.

“Transaminase” or “aminotransferase” are used interchangeably herein to refer to a polypeptide having an enzymatic capability of reversibly transferring an amino group (NH₂), a pair of electrons, and a proton from a primary amine to a carbonyl group (C═O) of an acceptor molecule. As would be understood by a skilled person, an enzyme having transaminase activity may also be capable of other reactions under different reaction conditions and different substrates, such as dehalogenation/deamination. The present invention includes all such enzymes.

Transaminases as used herein include naturally occurring (wild type) transaminase as well as non-naturally occurring engineered polypeptides generated by human manipulation. In one embodiment, an engineered transaminase polypeptide comprising an amino acid sequence having at least 80%, 85%, 90%, or 95% sequence identity to the amino acid sequence set out in SEQ ID NO: 2 or a functional fragment thereof, wherein the amino acid sequence comprises the feature that X124 is isoleucine (I) is disclosed. In one embodiment, an engineered transaminase polypeptide comprising an amino acid sequence having at least 80%, 85%, 90%, or 95% sequence identity to the amino acid sequence set out in SEQ ID NO: 2 or a functional fragment thereof, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 2, and wherein X124 is isoleucine (I) is disclosed.

In one embodiment, a transaminase polypeptide comprising an amino acid sequence having at least 80%, 85%, 90%, 95%, or 100% sequence identity to the amino acid sequence set forth in SEQ ID NOs: 4, 6, 8, 10, 14, 16, 18, or 20 is disclosed. In one embodiment, a transaminase polynucleotide comprising a polynucleotide sequence having at least 80%, 85%, 90%, 95%, or 100% sequence identity to the polynucleotide sequence set forth in SEQ ID NOs: 3, 5, 7, 9, 13, 15, 17, or 19 is disclosed.

In one aspect, the present invention provides an efficient process for the preparation of an asymmetric compound of Formula I:

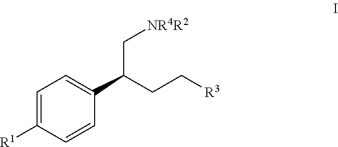

wherein:
R¹ is a leaving group, a halogen, a protected amino group, —NO₂, or —OH or its protected form;
R² is H;
R³ is —COOR⁵, —CH₂R⁶, or a protected aldehyde; or
R² and R³ are combined to form

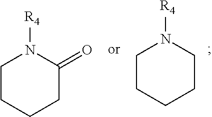

R⁴ is H or an amine protecting group;
R⁵ is C₁₋₆ alkyl, C₃₋₁₀ cycloalkyl, C₄₋₁₀ heterocyclyl, aryl, or heteroaryl; and,
R⁶ is a leaving group or —OH or its protected form; the process comprising contacting a compound of Formula II:

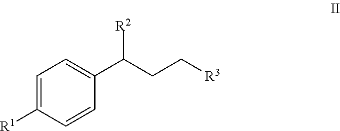

wherein:
R^{1′} is a leaving group, a halogen, a protected amino group, NO₂, or OH or its protected form;
R^{2′} is an aldehyde or an aldehyde equivalent; and,
R^{3′} is —COOR⁵, —CH₂R⁶, or a protected aldehyde; or
R^{2′} and R^{3′} are combined to form

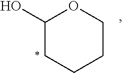

where * represents the point of attachment;
with the engineered transaminase polypeptide of any one of the preceding embodiments as disclosed herein.

“Alkyl” represents a saturated, straight or branched hydrocarbon moiety having the specified number of carbon atoms. The term “(C₁-C₆)alkyl” refers to an alkyl moiety containing from 1 to 6 carbon atoms. Exemplary alkyls include, but are not limited to, methyl, ethyl, n-propyl, isopropyl, n-butyl, isobutyl, s-butyl, t-butyl, pentyl, and hexyl. In some embodiments, “Me” refers to a methyl group. When the term “alkyl” is used in combination with other substituent groups, the term “alkyl” is intended to encompass a divalent straight or branched-chain hydrocarbon radical, wherein the point of attachment is through the alkyl moiety.

“Aryl” refers to a monocyclic or bicyclic, hydrocarbon, aromatic radical. Aryl includes, for example, phenyl and naphthyl. An aryl group may contain 6 to 14 carbon atoms. The term “heteroaryl” refers to a group or moiety comprising an aromatic monovalent monocyclic or bicyclic radical, containing 5 to 10 ring atoms, including at least one heteroatom independently selected from nitrogen, oxygen and sulfur. This term also encompasses bicyclic heterocyclic-aryl compounds containing an aryl ring moiety fused to a heterocycloalkyl ring moiety, containing 5 to 10 ring atoms, including at least one heteroatom independently selected from nitrogen, oxygen and sulfur. Exemplary groups include, but are not limited to furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 5-membered “heteroaryl” groups include furanyl, thienyl, pyrrolyl, imidazolyl, pyrazolyl, triazolyl, tetrazolyl, thiazolyl, oxazolyl, isoxazolyl, oxadiazolyl, thiadiazolyl, and isothiazolyl. Examples of 6-membered “heteroaryl” groups include oxo-pyridyl, pyridinyl, pyridazinyl, pyrazinyl, and pyrimidinyl. Examples of 6,6-fused “heteroaryl” groups include quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 6,5-fused “heteroaryl” groups include benzofuranyl, benzothienyl, benzimidazolyl, benzthiazolyl, indolizinyl, indolyl, isoindolyl, and indazolyl.

The term “cycloalkyl” refers to a non-aromatic, saturated, monocyclic, hydrocarbon ring containing the specified number of carbon atoms. The term “(C₃-C₆)cycloalkyl” refers to a non aromatic cyclic hydrocarbon ring having from three to six ring carbon atoms. Exemplary “(C₃-C₆)cycloalkyl” groups useful in the present invention include cyclopropyl, cyclobutyl, cyclopentyl, and cyclohexyl. Examples of “(C₃-C₆)cycloalkyl(C₁-C₄)alkyl-” groups useful in the present invention include, but are not limited to, cyclobutylmethyl, cyclopentylmethyl, cyclohexylmethyl, cyclobutylethyl, cyclopentylethyl, and cyclohexylethyl.

“Halogen” and “halo” represent fluoro, chloro, bromo, or iodo substituents.

The term “heterocycle” or “heterocyclyl,” as used herein, is intended to mean a 3- to 10-membered aromatic or nonaromatic heterocycle containing from 1 to 4 heteroatoms selected from the group consisting of O, N and S, and includes bicyclic groups. For the purposes of this invention, the term “heterocyclic” is also considered to be synonymous with the terms “heterocycle” and “heterocyclyl” and is understood as also having the definitions set forth herein. “Heterocyclyl” therefore includes the above mentioned heteroaryls, as well as dihydro and tetrahydro analogs thereof. Further examples of “heterocyclyl” include, but are not limited to the following: azetidinyl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxooxazolidinyl, oxazolyl, oxazoline, oxopiperazinyl, oxopyrrolidinyl, oxomorpholinyl, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrofuranyl, tetrahydrothiopyranyl, tetrahydroisoquinolinyl, tetrazolyl, tetrazolopyridyl, thiadiazolyl, thiazolyl, thienyl, triazolyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dioxidothiomorpholinyl, methylenedioxybenzoyl, tetrahydrofuranyl, and tetrahydrothienyl, and N-oxides thereof. Attachment of a heterocyclyl substituent can occur via a carbon atom or via a heteroatom.

“Hydroxy” or “hydroxyl” is intended to mean the radical —OH.

“Leaving group” is defined as a term that would be understood by one of ordinary skill in the art; that is, a group on a carbon where, upon reaction, a new bond is to be formed and the carbon loses the group upon formation of the new bond. A typical example employing a suitable leaving group is a nucleophilic substitution reaction, e.g., on a sp³ hybridized carbon (S_{N}2 or S_{N}1), e.g., where the leaving group is a halide, such as a bromide, the reactant might be benzyl bromide. Another typical example of such a reaction is a nucleophilic aromatic substitution reaction (SNAr). Another example is an insertion reaction (for example by a transition metal) into the bond between an aromatic reaction partner bearing a leaving group followed by reductive coupling. “Leaving group” is not limited to such mechanistic restrictions. Examples of suitable leaving groups include halogens (fluorine, chlorine, bromine or iodine), optionally substituted aryl or alkyl sulfonates, phosphonates, azides and —S(O)₀₋₂R where R is, for example optionally substituted alkyl, optionally substituted aryl, or optionally substituted heteroaryl. Those of skill in the art of organic synthesis will readily identify suitable leaving groups to perform a desired reaction under different reaction conditions. Non-limiting characteristics and examples of leaving groups can be found, for example in Organic Chemistry, 2nd ed., Francis Carey (1992), pages 328-331; Introduction to Organic Chemistry, 2d ed., Andrew Streitwieser and Clayton Heathcock (1981), pages 169-171; and Organic Chemistry, 5th Ed., John McMurry, Brooks/Cole Publishing (2000), pages 398 and 408.

“Protecting group” refers to a group of atoms that mask, reduce or prevent the reactivity of the functional group when attached to a reactive functional group in a molecule. Typically, a protecting group may be selectively removed as desired during the course of a synthesis. Examples of protecting groups can be found in Wuts and Greene, “Greene's Protective Groups in Organic Synthesis,” 4^{th} Ed., Wiley Interscience (2006), and Harrison et al., Compendium of Synthetic Organic Methods, Vols. 1-8, 1971-1996, John Wiley & Sons, NY. Functional groups that can have a protecting group include, but are not limited to, hydroxy, amino, and carboxy groups.

Representative amine protecting groups include, but are not limited to, formyl, acetyl (Ac), trifluoroacetyl, benzyl (Bn), benzoyl (Bz), carbamate, benzyloxycarbonyl (“CBZ”), p-methoxybenzyl carbonyl (Moz or MeOZ), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”), p-methoxybenzyl (PMB), tosyl (Ts) and the like.

Representative hydroxyl protecting groups include, but are not limited to, mesylate (SO₂Me), those where the hydroxyl group is either acylated (e.g., methyl and ethyl esters, acetate or propionate groups or glycol esters) or alkylated such as benzyl and trityl ethers, as well as alkyl ethers, tetrahydropyranyl ethers, trialkylsilyl ethers (e.g., TMS or TIPS groups) and allyl ethers. Other protecting groups can be found in the references noted herein.

A “protected aldehyde” is defined as the term would be understood by one of ordinary skill in the art; that is, the aldehyde is protected with a group such that it may be converted under assays conditions to an unprotected aldehyde. Examples of protected aldehydes include, but are not limited to, an acetal or hemiacetal which can be converted into a free aldehyde group by treatment with acids (organic or inorganic acids), such as acetal groups formed with a polyalcohol such as propane diol or ethylene glycol, or hemiacetal groups in a sugar or in a sugar-related compound such as an aldose sugar, e.g., glucose or galactose. Further examples of protected aldehydes are imino groups (e.g., ═NH groups), which give aldehyde groups upon treatment with acids; thioacetal or dithioacetal groups (e.g., C(SR)₂ groups wherein R may be an alkyl radical), which give aldehyde groups upon treatment with mercury salts; oxime groups (e.g., ═NOH groups), which give aldehyde groups upon treatment with acids; hydrazone groups (e.g., ═N—NHR groups wherein R may be an alkyl radical), which give aldehyde groups upon treatment with acids; and imidazolone or imidazolidine groups or benzothiazole or dihydrobenzothiazole groups, which give aldehydes upon hydrolysis, e.g., with acid.

### EXAMPLES

Many modifications and variations of the present disclosure are possible in light of the above teachings. It is therefore to be understood that, within the scope of the appended claims, a skilled person in the art would recognize that the invention may be practiced otherwise than as specifically described. The illustrative embodiments and examples should not be construed as limiting the invention.

### Example 1: Synthesis and Optimization of Engineered Transaminase Polypeptides

The DNA sequence of SEQ ID NO: 1 and polypeptide sequence of SEQ ID NO: 2 of the present disclosure correspond to the DNA sequence of SEQ ID NO: 179 and polypeptide sequence of SEQ ID NO: 180 of WO 2014/088984 A1, published Jun. 12, 2014. The engineered transaminase polypeptide of SEQ ID NO: 2 has the following 28 amino acid differences relative to the wild-type Arthrobacter sp. KNK168 polypeptide sequence (GenBank accession: BAK39753.1; GI:336088341): A2S; A5H; S8P, Y60F, L61Y, H62T, V65A, D81G, M94I, I96L, F122I, S124I, G136W, A169L, V199I, A209L, G215F, G217N, S223P, L269P, L273Y, T282S, A284G, P297S, 1306V, and S321P.

SEQ ID NO: 1 was cloned into the pCK110900 vector system (see e.g., U.S. Patent Application Publication No. 2006/0195947 A1), which is under the control of a lac promoter. This expression vector also contains the Pl5a origin of replication and the chloramphenicol resistance gene. Resulting plasmids were transformed into E. coli W3110 using standard methods and expressed as described in Example 2.

### Example 2: High-Throughput Screening for Identification of Variants of the Arthrobacter Spp. Aminotransferase Capable of Converting Lactol Substrate to Amine

### A) Library Preparation of Mutant Libraries

The gene encoding Arthrobacter spp aminotransferase (SEQ ID NO: 1), constructed as described in Example 1, was mutagenized and the population of altered DNA molecules was used to transform a suitable E. coli host strain.

### B) High-Throughput Growth and Expression of Mutant Libraries

Recombinant E. coli colonies carrying a gene encoding aminotransferase were picked using a Q-PIX molecular devices robotic colony picker (Genetix USA, Inc., Boston, MA) into 96-well shallow well microtiter plates containing in each well 180 μL LB Broth, 1% glucose and 30 μg/mL chloramphenicol (CAM). Plates were sealed with air-permeable nylon seals, and cells were grown overnight at 30° C. and 85% humidity with shaking at 200 rpm. A 20 μL aliquot of this culture was then transferred into 96-deep well plates containing 380 μL 2×YT broth and 30 g/mL CAM, supplemented with 100 μl pyridoxine. Again, plates were sealed with air-permeable nylon seals. After incubation of the deep-well plates at 30° C. and 85% humidity with shaking at 250 rpm for 2-3 hours, recombinant gene expression within the cultured cells was induced by addition of IPTG to a final concentration of 1 mM. The plates were then incubated at 30° C. and 85% humidity with shaking at 250 rpm for 18 hrs. Cells were pelleted by centrifugation (3738 RCF, 10 min, 4° C.), and pellets frozen at −80C for at least 2 hours.

### C) High-Throughput Assay Screening of Mutant Libraries for Identification of Improved Variants

Antibiotic resistant transformants were selected and processed to identify those expressing an aminotransferase with an improved ability to carry out the reaction below under desired reaction conditions.

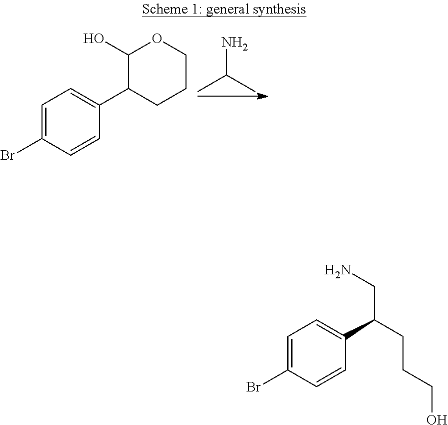

Cell pellets were thawed at room temperature for 30-120 minutes. Pellets were resuspended in 200 μL lysis solution containing buffer, 1 g/L lysozyme, and 0.5 g/L polymyxin B sulfate, and 0.25 g/L pyridoxal 5′-phosphate (PLP). The buffer and pH for the lysis solution varied according to screening conditions (see Table 3).

TABLE 3
Lysis Buffer

| Variants | Lysis Buffer |
|---|---|
| Variants 1, 2, 3, 5, 7 | 100 mM TEA, pH 8.5 |
| Variant 4 | 100 mM Sodium Phosphate, pH 9.5 |
| Variants 8, 9 | 100 mM KPi, pH 8.0 |

After sealing the plates with air-permeable nylon seals, they were shaken vigorously for 2 hours at room temperature. Cell debris was pelleted by centrifugation (3738 RCF, 10 min., 4° C.) and the clear supernatant assayed directly or stored at 4° C. until use. For screening of engineered aminotransferases substrates were prepared as follows: isopropylamine was pH adjusted to target pH condition in water, and lactol and morpholine dissolved in DMSO volume to target co-solvent condition after dilution. For all plates, lactol in DMSO and isopropylamine were added separately to a round-bottomed, shallow well plate, and diluted to final concentration by addition of lysate to start the reaction. Plates were heat-sealed with aluminum/polypropylene laminate heat seal tape at 165° C. for 3 seconds, and incubated at 650 rpm (INFORS Thermotron), for 20 hours. The conditions for the mutants are summarized below. Reactions were prepared for analysis as described in Example 3.

TABLE 4
Conditions used for each transaminase variant

| | Lactol | DMSO | IPM | Morpholine | | Temp | Lysate |
|---|---|---|---|---|---|---|---|
| | (g/L) | (%) | (M) | (eq) | pH | (°C) | (%) |
| Variant 1 | 30 | 5 | 1 | 5 | 8.5 | 45 | 10 |
| Variant 2 | 30 | 5 | 1 | 0.05 | 8.5 | 45 | 10 |
| Variant 3 | 50 | 20 | 1 | 0.05 | 8.5 | 45 | 25 |
| Variant 4 | 50 | 20 | 0.38 | 0.05 | 9.5 | 45 | 20 |
| Variant 5 | 30 | 30 | 1.6 | 0.05 | 8.5 | 25 | 20 |
| Variant 7 | 50 | 30 | 1.6 | 0.05 | 8.5 | 40 | 10 |
| Variant 8 | 50 | 30 | 1.6 | 0.05 | 8.5 | 40 | 5 |
| Variant 9 | 50 | 30 | 1.6 | 0.05 | 8.5 | 40 | 5 |

Variant 1 (SEQ ID NO: 4): 10% lysate, 30 g/L 3-(4-Bromophenyl)tetrahydro-2H-pyran-2-ol, 1M isopropylamine (pH8.5), 5% DMSO, 5 eq. morpholine, 45° C.
Variant 2 (SEQ ID NO: 6): 10% lysate, 30 g/L 3-(4-Bromophenyl)tetrahydro-2H-pyran-2-ol, 1M isopropylamine (pH8.5), 5% DMSO, 0.05 eq. morpholine, 45° C.
Variant 3 (SEQ ID NO: 8): 25% lysate, 50 g/L 3-(4-Bromophenyl)tetrahydro-2H-pyran-2-ol, 1M isopropylamine (pH8.5), 20% DMSO, 0.05 eq. morpholine, 45° C.
Variant 4 (SEQ ID NO: 10): 20% lysate, 50 g/L 3-(4-Bromophenyl)tetrahydro-2H-pyran-2-ol, 0.38M isopropylamine (pH9.5), 20% DMSO, 0.05 eq. morpholine, 45° C.
Variant 5 (SEQ ID NO: 12): 30% lysate, 30 g/L 3-(4-Bromophenyl)tetrahydro-2H-pyran-2-ol, 1.6M isopropylamine (pH9.5), 30% DMSO, 0.05 eq. morpholine, 25° C.
Variant 6 (SEQ ID NO: 14): 20% lysate, 30 g/L 3-(4-Bromophenyl)tetrahydro-2H-pyran-2-ol, 1.6M isopropylamine (pH8.5), 30% DMSO, 0.05 eq. morpholine, 25° C.
Variant 7 (SEQ ID NO: 16): 10% lysate, 50 g/L 3-(4-Bromophenyl)tetrahydro-2H-pyran-2-ol, 1.6M isopropylamine (pH8.5), 30% DMSO, 0.05 eq. morpholine, 40° C.
Variant 8 (SEQ ID NO: 18): 5% lysate, 50 g/L 3-(4-Bromophenyl)tetrahydro-2H-pyran-2-ol, 1.6M isopropylamine (pH8.5), 30% DMSO, 0.05 eq. morpholine, 40° C.
Variant 9 (SEQ ID NO: 20): 5% lysate, 50 g/L 3-(4-Bromophenyl)tetrahydro-2H-pyran-2-ol, 1.6M isopropylamine (pH8.5), 30% DMSO, 0.05 eq. morpholine, 40° C. Reactions run at higher pH allowed for better buffering of the reaction, however also had slower rates of reactions. Detection of undesired selectivity in the mutants could not be determined under these conditions. Unexpectedly, reactions run between pH 7.5-8.5 revealed mutants with any changes at position 1124 (particularly I124H—see Variant 5) had significant loss of selectivity. As a result, pH tolerance screening conditions were reverted to pH8.5 and challenged with increased co-solvent.

### Example 3: Production of Transaminase Powders—Shake Flask Procedure

A single microbial colony of E. coli containing a plasmid encoding a aminotransferase of interest was inoculated into 50 mL Luria Bertoni broth containing 30 μg/mL chloramphenicol and 1% glucose. Cells were grown overnight (at least 16 hours) in an incubator at 30° C. with shaking at 250 rpm.

The culture was diluted into 1000 mL of 2×YT containing 30 μg/mL chloramphenicol (supplemented with 0.1 mM pyridoxine) to give an approximate OD600 of 0.2 and allowed to grow at 30° C. with shaking at 250 rpm. Expression of the aminotransferase was induced by addition of isopropyl β D-thiogalactoside (IPTG) to a final concentration of 1 mM when the OD600 of the culture was 0.6 to 0.8. Incubation was then continued overnight (at least 16 hours).

Cells were harvested by centrifugation (3738 RCF, 20 min, 4° C.) and the supernatant discarded. Pellets were frozen for at least 2 hours at −80° C. Pellets were then thawed and resuspended at a ratio of 3 mL 100 mM Potassium phosphate buffer (pH7.5, supplemented with 500 μM PLP) per gram of final pellet mass (e.g., 10 g frozen pellet suspended in 30 mL buffer).

After resuspension, cells were filtered through 200 um mesh before passing twice through the microfluidizer at 12000 psig. Cell debris was removed by centrifugation (15,777 RCF, 40 min, 4° C.). The clarified lysate supernatant was collected, pooled, and lyophilized to provide a dry powder of crude aminotransferase enzyme.

### Example 4: Production of Transaminase Powders—Fermentation Procedure

An aliquot of frozen working stock (E. coli containing plasmid with the aminotransferase gene of interest) was removed from the freezer and allowed to thaw at room temperature. 300 μL of this working stock was inoculated into a primary seed stage of 250 ml M9YE broth (1.0 g/L ammonium chloride, 0.5 g/L of sodium chloride, 6.0 g/L of disodium monohydrogen phosphate, 3.0 g/L of potassium dihydrogen phosphate, 2.0 g/L of PROCELYS SPRINGER 0251 yeast extract, 1 L/L de-ionized water) containing 30 g/ml chloramphenicol and 1% glucose in 1 L flasks and allowed to grow at 37° C. with shaking at 200 rpm. When the OD600 of the culture was 0.5 to 1.0, the flasks were removed from the incubator and immediately used to inoculate a secondary seed stage.

A secondary seed stage was carried out in bench scale 5 L fermenters using 4 L of growth medium (0.88 g/L ammonium sulfate, 0.98 g/L of tri sodium citrate dihydrate; 12.5 g/L of dipotassium hydrogen phosphate, 6.25 g/L of potassium dihydrogen phosphate, 3.3 g/L of PROCELYS SPRINGER 0251 yeast extract, 0.083 g/L ferric ammonium citrate, 0.5 ml/L polypropylene glycol antifoam and 8.3 ml/L of a trace element solution, 1 L/L process water. Trace element solution contained 2 g/L of calcium chloride dihydrate, 2.2 g/L of zinc sulfate heptahydrate, 0.5 g/L manganese sulfate monohydrate, 1 g/L copper sulfate pentahydrate, 0.1 g/L ammonium molybdate tetrahydrate and 0.02 g/L sodium tetraborate decahydrate, 1 L/L de-ionized water). Growth medium was sterilized at 121° C. for 40 minutes. Post sterilization 40 ml/L of feed stock solution was added (feed stock solution contained 12 g/L ammonium sulfate, 5.1 g/L magnesium sulfate heptahydrate, 500 g/L dextrose monohydrate, 1 L/L process water, sterilized at 121° C. for 30 minutes). Fermenters were inoculated with 2 ml OD600 0.5-1.0 primary seed and supplemented with 30 g/ml chloramphenicol, incubated at 37° C., 300 rpm and 0.5 vvm aeration. When the OD600 of the culture was 0.5-1.0 the secondary seed was immediately transferred to a final stage fermentation.

The final stage fermentation was carried out at bench scale in 10 L fermenters using 6 L of growth medium (0.88 g/L ammonium sulfate, 0.98 g/L of tri sodium citrate dihydrate; 12.5 g/L of dipotassium hydrogen phosphate, 6.25 g/L of potassium dihydrogen phosphate, 3.3 g/L of Procelys Springer 0251 yeast extract, 0.083 g/L ferric ammonium citrate, 0.5 ml/L polypropylene glycol antifoam and 8.3 ml/L of a trace element solution, 1 L/L process water. Trace element solution contained 2 g/L of calcium chloride dihydrate, 2.2 g/L of zinc sulfate heptahydrate, 0.5 g/L manganese sulfate monohydrate, 1 g/L copper sulfate pentahydrate, 0.1 g/L ammonium molybdate tetrahydrate and 0.02 g/L sodium tetraborate decahydrate, 1 L/L de-ionized water). Growth medium was sterilized at 121° C. for 40 minutes. Post sterilization growth medium was supplemented with 0.035 g/L pyridoxine hydrochloride and 40 ml/L feed stock solution (feed stock solution contained 12 g/L ammonium sulfate, 5.1 g/L magnesium sulfate heptahydrate, 500 g/L dextrose monohydrate, 1 L/L process water, sterilized at 121° C. for 30 minutes).

Fermenters were inoculated with 500 ml OD600 0.5-1.0 secondary seed and incubated at 30° C. and 1.5 vvm aeration. Dissolved oxygen was controlled at 30% by variable speed agitation, pH was maintained at 7.0 by addition of 17.5% v/v ammonium hydroxide solution. Growth of the culture was maintained by addition of feed stock solution (12 g/L ammonium sulfate, 5.1 g/L magnesium sulfate heptahydrate, 500 g/L dextrose monohydrate, 1 L/L process water, sterilized at 121° C. for 30 minutes). After the culture reached an OD600 of 80+/−10 Isopropyl-β-D-thiogalactoside (IPTG) was added to a final concentration of 1 mM. The fermentation was continued for another 18 hours. At harvest the culture was chilled to 8° C. Cells were collected by centrifugation at 5000 G for 40 minutes in a Sorvall RC12BP centrifuge at 4° C. Harvested cell pellets were then frozen at −80° C. and stored until downstream processing and recovery.

### Harvest to Lyo Powder

The fermentation broth was buffered to pH 7.3-7.5 with dipotassium phosphate 6.88 g/L and monopotassium phosphate 1.43 g/L. Once salts had dissolved and target pH range attained the broth was homogenised by mechanical lysis. Polyethyleneimine sulfate (Mn ~60,000, Mw 750,000) was added to the lysate to a final concentration of <0.6% w/w (amount of flocculant required was determined by offline titration). The flocculant treated lysate was clarified by centrifugation, the solid phase was discarded, and the clarified lysate supernatant was retained. The clarified lysate was concentrated ~10-fold by tangential flow filtration through a 30 kDa MWCO membrane. Pyridoxal 5′-phosphate (PLP) was then added to the concentrate, the mass added was proportional to the starting fermentation broth volume, 0.08 g PLP per L of fermentation broth. The PLP concentrate was then lyophilised. Lyophilised material was then milled to a homogenous powder.

### Example 5: High-Throughput Analytical Method for Identification of Variants of the Arthrobacter Spp. Aminotransferase Capable of Converting Lactol Substrate to Amine

### A) Achiral Analysis

Following overnight reaction, plates were removed from the incubator, seals removed, and reactions were diluted with an equivalent volume of acetonitrile. Plates were heat-sealed with aluminum/polypropylene laminate heat seal tape at 165° C. for 4 seconds, shaken for 10 min and then centrifuged at 3738 RCF for 10 min to sediment debris. 20 uL per well 2-fold diluted supernatant was further diluted either 10- or 20-fold (for 30 g/L or 50 g/L reaction respectively) in 50% acetonitrile in water in new shallow well polypropylene plates. Plates were again sealed, mixed, and analyzed by UPLC described below.

### B) Achiral UPLC Method to Qualitatively Determine Amine Product

Enzymatic conversion of the lactol substrate, (3-(4-Bromophenyl)tetrahydro-2H-pyran-2-ol), to an amine product (see Scheme 1) was determined using an Agilent 1290 UPLC equipped with an Agilent ZORBAX SB-C18 RRHD column (3.0×50 mm, 1.8 μm) using a gradient of 0.05% Trifluoroacetic Acid in Water (mobile phase A) and 0.05% Trifluoroacetic in Acetonitrile (mobile phase B) at a flow rate of 2 mL/min at a column temperature of 60° C. The gradient profile is shown below.

TABLE 5
Gradient Profile

| Time (min) | A (%) | B (%) |
|---|---|---|
| 0.1 | 90 | 10 |
| 1 | 40 | 60 |
| 1.3 | 0 | 100 |
| 1.5 | 0 | 100 |
| 1.51 | 90 | 10 |
| 2 | 90 | 10 |

Compound elution was monitored at 214 nm, with DMSO eluting at ~0.125 min, followed by amine product at ~0.55 min, and lactol ~0.89 min. Four small impurity peaks elute between starting material and amine product.

### C) Derivatization of Amine Product by Marfey's Reagent:

1% MARFEY'S REAGENT (10 g/L in Acetonitrile) was prepared. 20 uL per well 2-fold diluted supernatant prepared as described in Example 3B was transferred into new 96 deep-well plate, followed by 30 μL 1 M NaHCO₃, then 200 μL 1% MARFEY'S REAGENT. Plates were sealed and incubated at 40° C., 850 rpm (INFORS Thermotron), for 1 hour. Derivatization was quenched by addition of equal volume 1:9 2N HCl:Acetonitrile. Plates were mixed for 5 minutes then centrifuged at 3738 RCF for 10 min to sediment debris. Seals were removed, and 40 μL supernatant was transferred to 160 μL acetonitrile in new shallow well polypropylene plates. Plates were again sealed, mixed, and analyzed by UPLC.

### D) Chiral UPLC Method to Qualitatively Determine Selectivity for Amine Product

High-throughput Quantification of product enantiomers was determined using an Agilent 1290 UPLC equipped with an Agilent ZORBAX SB-C18 RRHD column (3.0×50 mm, 1.8 μm) using a gradient of 0.05% Trifluoroacetic Acid in Water (mobile phase A) and 0.05% Trifluoroacetic in Acetonitrile (mobile phase B) at a flow rate of 2 mL/min at a column temperature of 60° C. The gradient profile is shown below.

TABLE 6
Gradient Profile

| Time (min) | A (%) | B (%) |
|---|---|---|
| 6 | 70 | 30 |
| 6.5 | 0 | 100 |
| 7 | 0 | 100 |
| 7.1 | 70 | 30 |
| 8 | 70 | 30 |

Compound elution was monitored at 340 nm, with desired product eluting at ~4.89 min, and undesired at ~5.15 min.

TABLE 7
Relative activity data

| | Representative | | Activity (Fold | |
|---|---|---|---|---|
| | Mutations | Number of | improvement | |
| SEQ ID NO: | relative to SEQ | Residue | compared to | Selectivity |
| (nt/aa) | ID NO: 2 | Differences | Control) | (% e.e.) |
| 1/2 (Control) | N/A | — | — | >99 |
| 3/4 (Variant 1) | Y61C | 1 | 2.4 | >99 |
| 5/6 (Variant 2) | P48R; V69C; | 5 | 5.4 | >99 |
| | I94C; I196R; | | | |
| | S297R | | | |
| 7/8 (Variant 3) | C61N | 1 | 8.1 | >99 |
| 9/10 (Variant 4) | C69T | 1 | 13.5 | >99 |
| 11/12 (Variant 5) | I124H | 1 | 41.7 | <80 |
| 13/14 (Variant 6) | N61M | 1 | 24.0 | >99 |
| 15/16 (Variant 7) | E137T | 1 | 27.1 | >99 |
| 17/18 (Variant 8) | I140Q; I199T; | 3 | 38.5 | >99 |
| | T202C | | | |
| 19/20 (Variant 9) | F3H; H5I; 197E; | 4 | 53.5 | >99 |
| | P269C | | | |

TABLE 8
Summary of active amino acid mutations in each variant

| Variants | Reference | Active Mutations compared to Control |
|---|---|---|
| Control | SEQ ID NO: 2 | None |
| Variant 1 | SEQ ID NO: 2 | Y61C |
| Variant 2 | SEQ ID NO: 2 | P48R; Y61C; V69C; I94C; I196R; S297R |
| Variant 3 | SEQ ID NO: 2 | P48R; Y61N; V69C; I94C; I196R; S297R |
| Variant 4 | SEQ ID NO: 2 | P48R; Y61N; V69T; I94C; I196R; S297R |
| Variant 5 | SEQ ID NO: 2 | P48R; Y61N; V69T; I94C; I124H; I196R; S297R |
| Variant 6 | SEQ ID NO: 2 | P48R; Y61M; V69T; I94C; I196R; S297R |
| Variant 7 | SEQ ID NO: 2 | P48R; Y61M; V69T; I94C; E137T; I196R; S297R |
| Variant 8 | SEQ ID NO: 2 | P48R; Y61M; V69T; I94C; E137T; I140Q; I196R; I199T; |
| | | T202C; S297R |
| Variant 9 | SEQ ID NO: 2 | F3H; H5I; P48R; Y61M; V69T; I94C; I97E; E137T; I140Q; |
| | | I196R; I199T; T202C; P269V; S297R |

Table 7 provides that all variants showed increased activities while maintaining the selectivity of greater than 99% e.e., except Variant 5 carrying mutations to position 1124. This was surprising and unexpected to the inventors of the present disclosure as any changes at position 1124 (particularly I124H—see variant 5) had significant loss of selectivity. This loss of selectivity was present in all tested sample variants when the I124H substitution was introduced (see Table 9).

TABLE 9
Effect of amino acid substitution at position I124 on selectivity

| | | Selectivity |
|---|---|---|
| | Active Mutations relative to SEQ ID NO: 10 | (% e.e.) |
| Sample 1 | I124Q | <94 |
| Sample 2 | I124A | <92 |
| Sample 3 | I124S | <95 |
| Sample 4 | I124A | <92 |
| Sample 5 | I124H | <95 |
| Sample 6 | I124H | <95 |
| Sample 7 | I124A | <93 |
| Sample 8 | I124H | <94 |
| Sample 9 | I124S | <95 |
| Sample 10 | I124H; W136F; E137T; V152C; F215C; R297S; Q329H | <90 |
| Sample 11 | N61Y; P99C; I124T; V152C; Q329H | <90 |
| Sample 12 | P99C; I124T; T134S; E137T; F215C; R297S | <89 |
| Sample 13 | I124T; W136F; E137T | <89 |
| Sample 14 | I124H; W136F; E137T; T178S; F215C | <85 |
| Sample 15 | I124H; W136F; T178S; R297S | <80 |
| Sample 16 | P99C; I124T; R297S | <93 |
| Sample 17 | C94I; I124H; V152C; R297S | <95 |

Screening conditions for high throughput characterization of enantioselectivity (Table 8) differed from those described in Example 2. Reactions were not progressed to completion, therefore enantioselectivity was not quantitative and differences can be seen in the values reported in Table 7. Reactions were sufficient to detect perturbances in enantioselectivity to enable selection of alternative variants to progress.

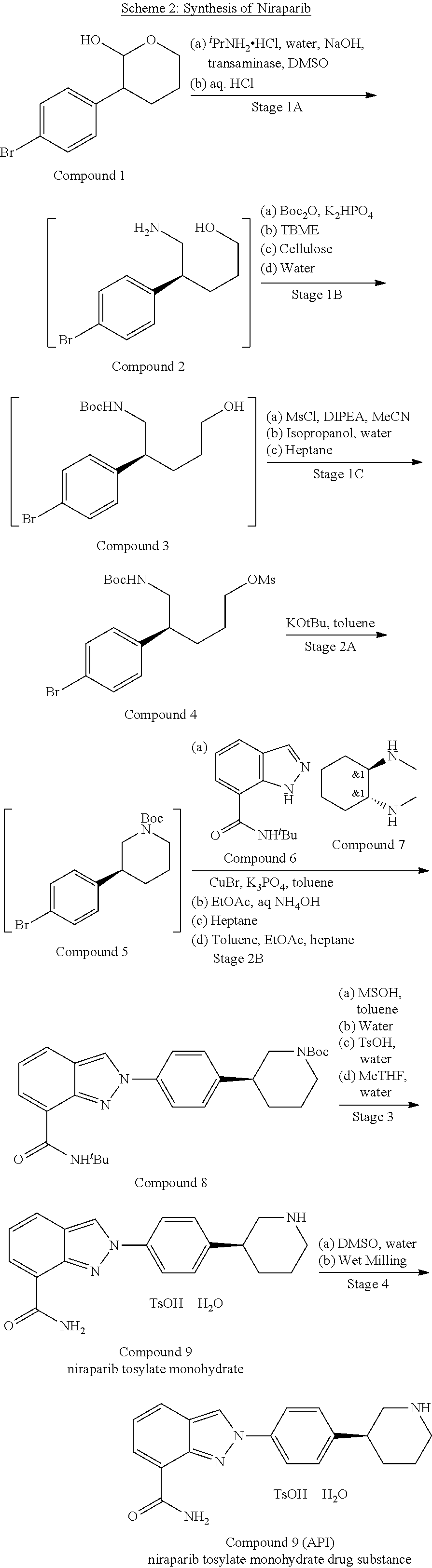

### Example 6: Chemical Process

### 1) Preparation of Compound 4

Stage 1A: To a stirred solution of isopropylamine hydrochloride in water at 25° C. is added aqueous 1M sodium hydroxide solution to adjust the pH to 8.5. Lyophilised transaminase is added and stirred for at least 15 minutes. A solution of Compound 1 in DMSO is added to the mixture. The pH is adjusted to 8.6 with aqueous 1M sodium hydroxide solution. The mixture is then heated to 44° C. and stirred for 21 hours. The reaction is cooled to 25° C. and the pH is adjusted to pH 2 using aqueous concentrated HCl solution and stirred for 1.5 hours to denature the enzyme.

Stage 1B: To the stirred solution afforded at Stage 1A is added K₂HPO₄, followed by liquid BOC anhydride. Stirring is continued. Water is added followed by TBME, which is then stirred for at least 10 minutes before being allowed to settle. The separated aqueous layer is removed. Cellulose is added and stirred for at least 15 min before being filtered. The enzyme filter cake is washed with TBME before the solids are discarded. The combined organics are washed twice with water, the separated aqueous layers being removed each time. The organic solution is concentrated to ca 5 vol under vacuum, and then solvent switched to acetonitrile.

Stage 1C: DIPEA is added to the solution, cooled to 0° C., and mesyl chloride is added over 1 hour while maintaining the contents temperature at <5° C. Following the final addition, the mixture is stirred for at least 30 minutes. Isopropanol is added and the contents temperature adjusted to 20° C. before water is added over at least 10 minutes. The mixture is seeded with Compound 4 and aged for 2 hours. Water is added over at least 1.5 hours, stirred for at least 2 hours and filtered to dry land. The filter cake is washed successively with two isopropanol/water (1:1 v/v) cake washes, followed by pre-chilled isopropanol and finally heptane. The cake is deliquored and dried in a vacuum oven at 55° C.

### 2) Preparation of Compound 8

Stage 2A: Preparation of Compound 5. Toluene is charged to the reactor at 25±5° C. with stirring. Compound 7 is charged to the reactor followed by potassium tert-butoxide. The mixture is stirred at 25±5° C. until reaction is complete.

Stage 2B: Preparation of Compound 8. Tripotassium phosphate (K3PO4), Compound 6 and copper(I) bromide are charged to the reactor followed by trans-N1,N2-dimethylcyclohexane-1,2-diamine (DMCyDA). The mixture is heated to reflux (105±10° C.) and stirred at this temperature until reaction is complete. The mixture is cooled to 45±5° C. then ethyl acetate is charged followed by water and 20% v/v aqueous NH₃. The layers are separated then water and 20% v/v aqueous NH₃ is added to the organic layer. The layers are separated. The organic layer is concentrated by distillation then seeded at 45±5° C. The mixture is aged then heptane is added. The resulting slurry is cooled to 0±5° C. and aged for NLT 16 hrs. The mixture is filtered then the cake is washed with toluene/EtOAc/heptane mixture. The product is dried at 60±5° C. under vacuum to afford Compound 8.

### 3) Preparation of Compound 9

Methanesulfonic acid (MsOH) is added to a slurry of Compound 8 in toluene over at least 3 hours whilst maintaining the temperature below 40° C. The resulting biphasic solution is stirred at 38±5° C. for 3 hours. The reactor contents are then cooled to 0±5° C. Water is added while maintaining the temperature below 30° C. The aqueous and organic phases are then separated. The aqueous phase is washed with toluene and then filtered. A portion of the solution of p-Toluenesulfonic acid is added to the filtrate, followed by Compound 9 seed (made by the same process described herein). The remaining p-Toluenesulfonic acid solution is added at 20±5° C., the slurry is then cooled and stirred at 0±5° C. for NLT 4 hours. The slurry is filtered, washed by four water/MeTHF (9:1 v/v) cake washes and then the filter cake is dried under vacuum a NGT 40° C.

### 4) Purification of Compound 9

Compound 9 is added to DMSO and heated to 33° C. to dissolve. This solution is charged into water over 2 hours with agitation. After a one-hour hold, the suspension is wet-milled. The resulting suspension is heated to 65° C. held for seven hours and then cooled to 5° C. The slurry is isolated and washed using water. The material is dried with periodic agitation up to 40° C.

Impurity (Compound 10) is formed as a by-product in the Stage 3 chemistry when methanesulfonic acid is added to a slurry of Compound 8 in toluene.

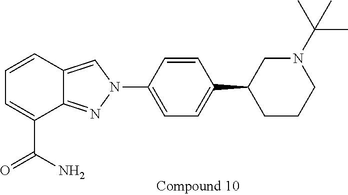 [SMILES: CC(C)(C)N1CCC[C@@H](c2ccc(-n3cc4cccc(C(N)=O)c4n3)cc2)C1]

Compound 10 is controlled to not more than 0.10% w/w in niraparib tosylate monohydrate drug substance.

### Example 7: Structural Characterization

7.1.

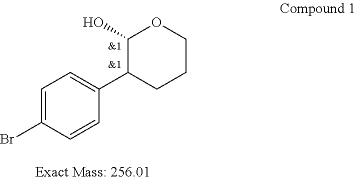 [SMILES: O[C@@H]1OCCCC1c1ccc(Br)cc1]

The structure of Compound 1, a crystalline solid, has been established through analysis by mass spectrometry and NMR spectroscopy.

LCMS (2.0 min ASAP): Rt=N/A, [MH]⁻=255.0026

¹H NMR (CHLOROFORM-d, 700 MHz): δ (ppm) 7.41-7.48 (m, 3H), 7.20 (d, J=8.4 Hz, 1H), 7.14 (d, J=8.4 Hz, 2H), 6.45-6.46 (m, 1H), 5.20 (d, J=2.7 Hz, 1H), 4.76 (d, J=7.9 Hz, 1H), 4.10 (td, J=4.5, 2.5 Hz, 1H), 3.61-3.66 (m, 1H), 3.42-3.42 (m, 1H), 2.95 (dt, J=12.4, 3.1 Hz, 1H), 2.59 (ddd, J=11.4, 7.7, 4.1 Hz, 1H), 1.98-2.07 (m, 1H), 1.78-1.82 (m, 1H), 1.75 (br s, 1H), 1.70-1.77 (m, 1H), 1.68-1.75 (m, 1H), 1.62-1.66 (m, 1H).

7.2.

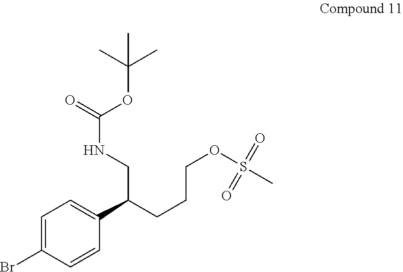 [SMILES: CC(C)(C)OC(=O)NC[C@@H](CCCOS(C)(=O)=O)c1ccc(Br)cc1]

The structure of Compound 11, a crystalline solid, has been established through analysis by mass spectrometry and NMR spectroscopy.

LCMS (5.5 min Trifluoracetic acid): Rt=2.33 min, [MH]⁺=436.0786

¹H NMR (DMSO-d₆, 700 MHz): δ (ppm) 7.46-7.50 (m, J=8.3 Hz, 2H), 7.14-7.16 (m, J=8.4 Hz, 2H), 6.80 (br t, J=5.7 Hz, 1H), 4.09-4.14 (m, 2H), 3.12 (s, 4H), 3.02-3.09 (m, 1H), 2.70-2.77 (m, 1H), 1.67-1.78 (m, 1H), 1.46-1.56 (m, 2H), 1.36-1.45 (m, 1H), 1.32 (s, 9H)

7.3.

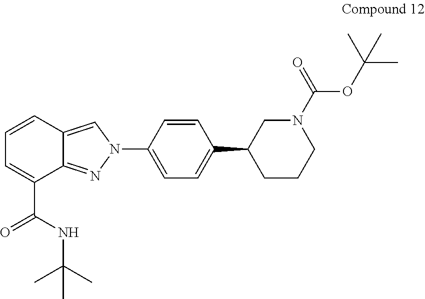 [SMILES: CC(C)(C)NC(=O)c1cccc2cn(-c3ccc([C@@H]4CCCN(C(=O)OC(C)(C)C)C4)cc3)nc12]

The structure of Compound 12, a crystalline solid, has been established through analysis by mass spectrometry and NMR spectroscopy.

LCMS (5.5 min Trifluoracetic acid): Rt=3.01 min, [MH]⁺=477.2869

¹H NMR (CHLOROFORM-d, 700 MHz): δ (ppm) 9.90 (br s, 1H), 8.54 (s, 1H), 8.21 (d, J=7.1 Hz, 1H), 7.91 (d, J=8.3 Hz, 1H), 7.83 (d, J=8.4 Hz, 2H), 7.44 (d, J=8.4 Hz, 2H), 7.26-7.30 (m, 1H), 4.22 (br d, J=12.2 Hz, 1H), 4.16 (br dd, J=13.4, 1.0 Hz, 1H), 2.84-2.87 (m, 1H), 2.82-2.89 (m, 1H), 2.78-2.85 (m, 1H), 2.11 (br d, J=12.7 Hz, 1H), 1.86 (dt, J=13.3, 2.9 Hz, 1H), 1.72 (br dd, J=11.9, 3.0 Hz, 1H), 1.64 (dt, J=12.9, 3.7 Hz, 1H), 1.60 (s, 9H), 1.50 (s, 9H).

7.4.

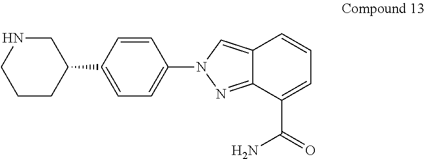 [SMILES: NC(=O)c1cccc2cn(-c3ccc([C@@H]4CCCNC4)cc3)nc12]

The structure of Compound 13, a crystalline solid, has been established through analysis by mass spectrometry and NMR spectroscopy.

LCMS (50 min Trifluoracetic acid): Rt=17.0 min, [MH]⁺=321.1711

¹H NMR (DMSO-d₆, 700 MHz): δ (ppm) 7.46-7.50 (m, J=8.3 Hz, 2H), 7.14-7.16 (m, J=8.4 Hz, 2H), 6.80 (br t, J=5.7 Hz, 1H), 4.09-4.14 (m, 2H), 3.12 (s, 4H), 3.02-3.09 (m, 1H), 2.70-2.77 (m, 1H), 1.67-1.78 (m, 1H), 1.46-1.56 (m, 2H), 1.36-1.45 (m, 1H), 1.32 (s, 9H).

1. An engineered transaminase polypeptide comprising an amino acid sequence having at least 80%, 85%, 90%, or 95% sequence identity to the amino acid sequence set forth in SEQ ID NO: 2 or a functional fragment thereof, wherein the amino acid sequence comprises the feature that X124 is isoleucine (I).

2. The engineered transaminase polypeptide of claim 1, wherein the amino acid sequence comprises the feature that: at least one amino acid selected from the group consisting of amino acid X3, X5, X48, X61, X69, X94, X97, X137, X140, X196, X199, X202, X269, and X297 is substituted to a different amino acid to that found at the corresponding amino acid position in SEQ ID NO: 2.

3. (canceled)

4. The engineered transaminase polypeptide of claim 1, wherein X61 is cysteine (C), asparagine (N), or methionine (M), and/or wherein X69 is cysteine (C) or threonine (T).

5. The engineered transaminase polypeptide of claim 1, wherein the amino acid sequence comprises at least one feature selected from the group consisting of: X48 is substituted with arginine (R), X94 is substituted with C, X196 is substituted with R, X297 is substituted with R, and X137 is substituted with T.

6. The engineered transaminase polypeptide of claim 1, wherein the amino acid sequence comprises at least one feature selected from the group consisting of: X140 is substituted with glutamine (Q), X199 is substituted with T, and X202 is substituted with C.

7. The engineered transaminase polypeptide of claim 1, wherein the amino acid sequence comprises at least one feature selected from the group consisting of: X3 is substituted with histidine (H), X5 is substituted with I, X97 is substituted with glutamate (E), and X269 is substituted with C or valine (V).

8. The engineered transaminase polypeptide of claim 1, wherein X61 is C.

9. The engineered transaminase polypeptide of claim 1, wherein the amino acid sequence comprises at least the following features: X48 is substituted with R, X61 is C, X69 is C, X94 is substituted with C, X196 is substituted with R, and X297 is substituted with R.

10. The engineered transaminase polypeptide of claim 1, wherein the amino acid sequence comprises at least the following features: X48 is substituted with R, X61 is N, X69 is C, X94 is substituted with C, X196 is substituted with R, and X297 is substituted with R.

11. The engineered transaminase polypeptide of claim 1, wherein the amino acid sequence comprises at least the following features: X48 is substituted with R, X61 is N, X69 is T, X94 is substituted with C, X196 is substituted with R, and X297 is substituted with R.

12. The engineered transaminase polypeptide of claim 1, wherein the amino acid sequence comprises at least the following features: X48 is substituted with R, X61 is M, X69 is T, X94 is substituted with C, X196 is substituted with R, and X297 is substituted with R.

13. The engineered transaminase polypeptide of claim 1, wherein the amino acid sequence comprises at least the following features: X48 is substituted with R, X61 is M, X69 is T, X94 is substituted with C, X137 is substituted with T, X196 is substituted with R, and X297 is substituted with R.

14. The engineered transaminase polypeptide of claim 1, wherein the amino acid sequence comprises at least the following features: X48 is substituted with R, X61 is M, X69 is T, X94 is substituted with C, X137 is substituted with T, X140 is substituted with Q, X196 is substituted with R, X199 is substituted with T, X202 is substituted with C, and X297 is substituted with R.

15. The engineered transaminase polypeptide of claim 1, wherein the amino acid sequence comprises at least the following features: X3 is substituted with H, X5 is substituted with I, X48 is substituted with R, X61 is M, X69 is T, X94 is substituted with C, X97 is substituted with E, X137 is substituted with T, X140 is substituted with Q, X196 is substituted with R, X199 is substituted with T, X202 is substituted with C, X269 is substituted with V, and X297 is substituted with R.

16. The engineered transaminase polypeptide of claim 1, wherein the amino acid sequence corresponds to the sequence of SEQ ID NOs: 4, 6, 8, 10, 14, 16, 18 or 20.

17. A polynucleotide encoding the engineered transaminase polypeptide of claim 1.

18. The polynucleotide of claim 17, wherein polynucleotide sequence corresponds to the sequence of SEQ ID NOs: 3, 5, 7, 9, 13, 15, 17 or 19.

19. An expression vector comprising the polynucleotide of claim 17.

20. A host cell comprising the polynucleotide of claim 17 or the expression vector of claim 19.

21. A process for preparing an asymmetric compound of Formula I: 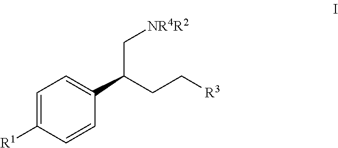
wherein:
R¹ is a leaving group, a halogen, a protected amino group, NO₂, or OH or its protected form;
R² is H;
R³ is —COOR⁵, —CH₂R⁶, or a protected aldehyde; or
R² and R³ are combined to form 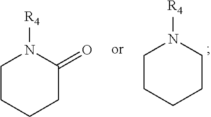
R⁴ is H or an amine protecting group;
R⁵ is C₁₋₆ alkyl, C₃₋₆ cycloalkyl, C₄₋₁₀ heterocyclyl, aryl, or heteroaryl; and,
R⁶ is a leaving group or OH or its protected form;
the process comprising contacting a compound of Formula II: 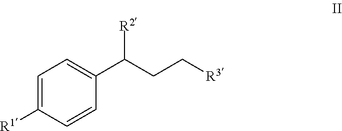
wherein:
R^{1′} is a leaving group, a halogen, a protected amino group, NO₂, or OH or its protected form;
R^{2′} is an aldehyde or an aldehyde equivalent; and
R^{3′} is —COOR⁵, —CH₂R⁶, or a protected aldehyde; or
R^{2′} and R^{3′} are combined to form 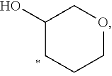
where * represents the point of attachment;
with an engineered transaminase polypeptide in the presence of a coenzyme and an amino donor.

22-61. (canceled)

62. A composition, comprising
i) niraparib tosylate monohydrate of Formula VII: 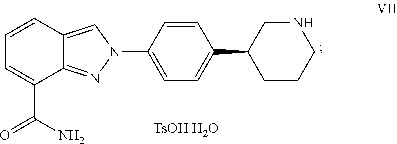
and
ii) less than 0.1 weight % of the compound of Formula VI, or a salt thereof,

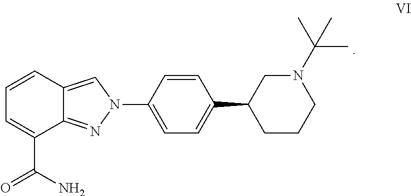 [SMILES: CC(C)(C)N1CCC[C@@H](c2ccc(-n3cc4cccc(C(N)=O)c4n3)cc2)C1]

63. (canceled)
